(19) United States
(12) Patent Application Publication
(10) Pub. No.: US 2009/0198324 A1
(43) Pub. Date: Aug. 6, 2009

(54) METHODS AND DEVICES FOR TREATMENT OF CARDIAC VALVES

(75) Inventor: Boris Orlov, Haifa (IL)

Correspondence Address: MARTIN D. MOYNIHAN d/b/a PRTSI, INC., P.O. BOX 16446, ARLINGTON, VA, 22215, US

(73) Assignee: Mor Research Applications Ltd., Tel-Aviv (IL)

(21) Appl. No.: 12/302,901

(22) PCT Filed: May 21, 2007
(86) PCT No.: PCT/IL2007/000615

§ 371 Date: Dec. 1, 2008

Related U.S. Application Data
(60) Provisional application No. 60/809,848, filed on Jun. 1, 2006.
(60) Provisional application No. 60/814,572, filed on Jun. 19, 2006.
(60) Provisional application No. 60/832,162, filed on Jul. 21, 2006.
(60) Provisional application No. 60/832,142, filed on Jul. 21, 2006.
(60) Provisional application No. 60/860,805, filed on Nov. 24, 2006.

(51) Int. Cl. A61F 2/24

(52) U.S. Cl. 623/2.37

(57) ABSTRACT

Disclosed are methods for treatment of cardiac valve including augmenting a cardiac leaflet with the help of a ring associated with a membrane. Also disclosed are methods for treatment of cardiac valves including augmenting the tissue surrounding a cardiac valve, for example with the help of a tubular or annular implant, allowing relocation of the valve. In embodiments, the methods of the present invention improve leaflet coaptation, which in embodiments is useful for treating conditions such as ischemic mitral regurgitation. Also disclosed are devices useful for implementing the methods of the present invention.

### RELATED APPLICATIONS

The present application gains benefit of the filing dates of U.S. patent application Nos. 60/809,848 filed 1 Jun. 2006; 60/814,572 filed 19 Jun. 2006; 60/832,142 filed 21 Jul. 2006; 60/832,162 filed 21 Jul. 2006 and 60/860,805 filed 24 Nov. 2006 all which are incorporated by reference as if fully set forth herein.

### FIELD AND BACKGROUND OF THE INVENTION

The present invention relates to the field of surgery and especially to methods and devices useful for augmenting cardiac valve leaflets or in augmenting tissue surrounding a cardiac valve, for example to allow relocation of the intact cardiac valve. Embodiments of the teachings of the present invention allow, for example, improving leaflet coaptation, for example in order to treat ischemic mitral regurgitation.

The human heart 10, depicted in cross sectional long axis view in FIG. 1, is a muscular organ that pumps deoxygenated blood through the lungs to oxygenate the blood and pumps oxygenated blood to the rest of the body by rhythmic contractions of four chambers.

After having circulated in the body, deoxygenated blood from the body enters the right atrium 12 through the vena cava 14. Right atrium 12 contracts, pumping the blood through a tricuspid valve 16 into the right ventricle 18. Right ventricle 18 contracts, pumping the blood through the pulmonary semi-lunar valve 20 into the pulmonary artery 22 which splits to two branches, one for each lung. The blood is oxygenated while passing through the lungs and reenters the heart to the left atrium 24.

Left atrium 24 contracts, pumping the oxygenated blood through the mitral valve 26 into the left ventricle 28. Left ventricle 28 contracts, pumping the oxygenated blood through the aortic semi-lunar valve 30 into the aorta 32. From aorta 32, the oxygenated blood is distributed to the rest of the body.

Physically separating left ventricle 28 and right ventricle 18 is interventricular septum 33. Physically separating left atrium 24 and right atrium 12 is an interatrial septum.

Mitral valve 26, depicted in FIG. 2A (top view) and in FIG. 2B (cross sectional long axis view) is defined by an approximately circular mitral annulus 34 that defines a mitral lumen 36. Attached to the periphery of mitral annulus 34 is an anterior leaflet 38 and a smaller posterior leaflet 40, leaflets 38 and 40 joined at commissures 41. Each leaflet is between about 0.8 and 2.4 mm thick and composed of three layers of soft tissue.

The typical area of mitral lumen 36 in a healthy adult is between 4 and 6 cm² while the typical total surface area of leaflets 38 and 40 is approximately 12 cm². Consequently and as depicted in FIG. 2B, leaflets 38 and 40 curve downwards into left ventricle 28 and coapt to accommodate the excess leaflet surface area, producing a coaptation surface 42 that constitutes a seal. The typical length of coaptation surface 42 in a healthy heart 10 of an adult is approximately 7-8 mm.

The bottom surface of anterior leaflet 38 and posterior leaflet 40 are connected to papillary muscles 44 at the bottom of left ventricle 28 by posterior chordae 46 and anterior chordae 48.

During diastole, left atrium 24 contracts to pump blood downwards into left ventricle 28 through mitral valve 26. The blood flows through mitral lumen 36 pushing leaflets 38 and 40 downwards into left ventricle 28 with little resistance.

During systole left ventricle 28 contracts to pump blood upwards into aorta 32 through aortic semi-lunar valve 30. Mitral annulus 34 contracts pushing leaflets 38 and 40 inwards and downwards, reducing the area of mitral lumen 36 by about 20% to 30% and increasing the length of coaptation surface 42. The pressure of blood in left ventricle 28 pushes against the bottom surfaces of leaflets 38 and 40, tightly pressing leaflets 38 and 40 together at coaptation surface 42 so that a tight leak-proof seal is formed. To prevent prolapse of leaflets 38 and 40 upwards into left atrium 24, papillary muscles 44 contract pulling the edges of leaflets 38 and 40 downwards through posterior chordae 46 and anterior chordae 48, respectively.

As is clear from the description above, an effective seal of mitral valve 26 is dependent on a sufficient degree of coaptation, in terms of length, area and continuity of coaptation surface 42. If coaptation surface 42 is insufficient or non-existent, there is mitral valve insufficiency, that is, regurgitation of blood from left ventricle 28 up into left atrium 24. A lack of sufficient coaptation may be caused by any number of physical anomalies that allow leaflet prolapse (e.g., elongated or ruptured chordae 46 and 48, weak papillary muscles 44) or prevent coaptation (e.g., short chordae 46 and 48, small leaflets 38 and 40).

Mitral valve insufficiency leads to many complications including arrhythmia, atrial fibrillation, cardiac palpitations, chest pain, congestive heart failure, fainting, fatigue, low cardiac output, orthopnea, paroxysmal nocturnal dyspnea, pulmonary edema, shortness of breath, and sudden death.

There are a number of pathologies that lead to a mitral valve insufficiency including collagen vascular disease, ischemic mitral regurgitation, myxomatous degeneration of leaflets 38 and 40 and rheumatic heart disease.

In ischemic mitral regurgitation (resulting, e.g., from myocardial infarction, chronic heart failure, or surgical or catheter revascularization), leaflets 38 and 40 and chordae 46 and 48 have normal structure and the mitral valve insufficiency results from altered geometry of left ventricle 28. As a result of ischemia, portions of the heart walls necrose. During healing, the necrotic tissue is replaced with unorganized tissue leading to remodeling of the heart which reduces coaptation through distortion of mitral annulus 34 and sagging of the outer wall of left ventricle 28 which displaces papillary muscles 44.

In FIGS. 3A (top view) and 3B (cross sectional long axis view), The reduction of coaptation resulting from ischemia is depicted for a mitral valve 26 of an ischemic heart 50 that has undergone mild remodeling and suffers from ischemic mitral regurgitation. In FIG. 3B is seen how an outer wall of left ventricle 28 sags outwards, displacing papillary muscles 44 downwards which, through chordae 46 and 48, pulls leaflets 38 and 40 downwards and apart, reducing coaptation. The incomplete closure of mitral valve 26 is seen in FIGS. 3A and 3B.

Initially, ischemic mitral regurgitation is a minor problem, typically leading only to shortness of breath during physical exercise due to the fact that a small fraction of blood pumped by left ventricle 28 is pumped into left atrium 24 and not through aortic semi-lunar valve 30, reducing heart capacity. To compensate for the reduced capacity, left ventricle 28 beats harder and consequently remodeling continues. Ultimately leaflet coaptation is entirely eliminated as leaflets 38 and 40 are pulled further and further apart, leading to more blood regurgitation, further increasing the load on left ventricle 28, and further remodeling. Ultimately, the left side of the heart fails and the person dies.

Apart from humans, mammals that suffer from mitral valve insufficiency include horses, cats, dogs, cows and pigs.

Currently, it is accepted to use open-heart surgical methods to improve mitral valve functioning by many different methods that force parts of the heart to adopt a shape that reduces some symptoms of improper valve function, including: modifying the subvalvular apparatus (e.g. lengthening the chordae) to improve leaflet coaptation; implanting an annuloplasty ring, e.g., as described in U.S. Pat. Nos. 3,656,185, 6,183,512 and 6,250,308 to force mitral valve annulus 34 into a normal shape; or implanting devices in the mitral valve to act as prosthetic leaflets, e.g., United States Patent applications published as US 2002/065554, US 2003/0033009, US 2004/0138745 or US 2005/0038509. It has been found that such methods often fail to provide sufficient long range improvement of valve function.

Surgical augmentation of a mitral valve anterior leaflet 38 for improving mitral valve leaflet coaptation for treating ischemic mitral valve regurgitation is taught by Kincaid et al (Kincaid E H, Riley R D, Hines M H, Hammon J W and Kon N D in Ann. Thorac. Surg. 2004, 78, 564-568). An incision is made in the anterior leaflet almost from commissure to commissure. The edges of a roughly elliptical patch of material (e.g., bovine pericardium, 1 cm wide, 3 cm long) are sutured to either side of the incision augmenting the anterior leaflet by an amount roughly equal to the surface area of the patch. Additionally, a flexible annuloplasty ring is implanted to reshape the mitral annulus. Although effective, such augmentation is considered a complex surgical procedure performed only by cardiac surgeons having above average skill.

It would be highly advantageous to have a way to restore cardiac valve function such as of a mitral valve by improving leaflet coaptation, to reduce mitral insufficiency, for example for treating subjects suffering from ischemic mitral valve regurgitation.

### SUMMARY OF THE INVENTION

Embodiments of the present invention successfully address at least some of the shortcomings of the prior art by providing methods and devices for the treatment of cardiac valves, which in embodiments improves cardiac valve leaflet coaptation, which may be useful in treating conditions, for example mitral insufficiency such as ischemic mitral regurgitation. In embodiments, the present invention also provides devices reminiscent of annuloplasty rings that allow procedures such as leaflet augmentation or cardiac valve relocation to be performed quickly with less dependence on the skill level or degree of exhaustion of the performing surgeon.

In a first aspect, the present invention provides for innovative methods and devices for leaflet augmentation. Embodiments of the present invention successfully address at least some of the shortcomings of the prior art by providing methods and apparatuses for reconstructing and realigning cardiac valve leaflets, for example mitral valve leaflets, some embodiments of which may be useful in treating conditions, for example mitral insufficiency such as ischemic mitral regurgitation. Generally, such apparatuses of the present invention can be considered as annuloplasty rings that are configured to support a leaflet-augmenting membrane. Generally, in embodiments such a device is deployed substantially as an annuloplasty ring, where a native leaflet is detached from the mitral valve annulus and secured to the leaflet augmenting membrane of the device, effectively lengthening the leaflet, which in embodiments restores or increases leaflet coaptation.

Thus, according to the teachings of the present invention, there is provided an annuloplasty apparatus comprising a substantially complete ring defining a ring lumen including an inner portion configured to be operatively associated with a lumen of an in vivo cardiac valve and an outer portion configured to be operatively associated with a periphery of the lumen of the cardiac valve, the annuloplasty apparatus further including a membrane functionally associated with the ring, the membrane at least partially covering the ring lumen around the entire periphery of the ring lumen in a plane substantially parallel to a plane passing radially through the ring.

In some embodiments, the membrane is continuous and substantially entirely covers the ring lumen.

In some embodiments, the membrane is provided with a membrane opening through the ring lumen. In some embodiments, the membrane opening is located substantially in the center of the ring lumen. In some embodiments, the membrane opening is located off-center of the ring lumen. In some embodiments, the membrane opening has an area of at least about 10% of the area of the ring lumen. In some embodiments, the membrane opening has an area of at least about 20% of the area of the ring lumen. In some embodiments, the membrane opening has an area of no more than about 80% of the area of the ring lumen.

In some embodiments, at least a portion of the ring includes a portion being substantially covered by the membrane. In some embodiments, the portion covered by the membrane includes the ring outer portion.

In some embodiments, the membrane covering ring outer portion is configured for securing proximate to a cardiac annulus and/or the periphery of a cardiac annulus.

In some embodiments, the membrane covering the ring outer portion is configured to be sutured to the valve periphery.

In some embodiments, the membrane encircles the ring so as to be functionally associated therewith.

In some embodiments, the membrane is secured to the ring so as to be functionally associated therewith.

In some embodiments, the membrane is secured to the ring by a member of the group consisting of sewing, adhesion, gluing, suturing, riveting and welding.

In some embodiments, the ring is configured to be sutured.

In some embodiments, the membrane is configured to be intra-operatively modified by at least one member of the group of processes consisting of cutting, bending, folding and suturing.

In some embodiments, the membrane comprises a tissue from an animal source such as a material from the group of materials consisting of serous tissue, pericardium, pleura, peritoneum and aortic leaflet.

In some embodiments, the animal source is a source from the group consisting of bovine, porcine, equine and human.

In some embodiments, the membrane is at least about 0.2 millimeters thick. In some embodiments, the membrane is no more than about 2 millimeters thick.

In embodiments, the ring is substantially similar to prior art annuloplasty rings and is fashioned from materials and in a manner as is known in the art of annuloplasty rings. In some embodiments, the ring comprises a material selected from a group consisting of nitinol, stainless steel shape memory materials, metals, synthetic biostable polymer, a natural polymer, an inorganic material, titanium, pyrolytic carbon, a plastic, a titanium mesh and polydimethylsiloxane.

In embodiments, a biostable polymer from which a ring is fashioned comprises a material from the group including a polyolefin, polyethylene, polytetrafluoroethylene (Teflon®), and polycarbonate synthetic, a polyurethane, a fluorinated polyolefin, a chlorinated polyolefin, a polyamide, an acrylate polymer, an acrylamide polymer, a vinyl polymer, a polyacetal, a polycarbonate, a polyether, an aromatic polyester, a polyether (ether ketone), a polysulfone, a silicone rubber (e.g., Silastic by Dow-Coming Corporation, Midland, Mich., U.S.A.), a thermoset material, or a polyester (ester imide, for example Dacron® by Invista, Wichita, Kans., U.S.A.) and/or combinations thereof.

In some embodiments, the ring comprises a material having a property selected from the group consisting of: flexible, plastic, elastic and rigid.

In some embodiments, the ring has height of no more than about 5.0 millimeters.

In some embodiments, the ring has height of at least about 1.0 millimeter.

According to the teachings of the present invention, there is also provided a method for performing an annuloplasty procedure in a heart (human or non-human, such as dog, cat, pig, horse or cow), comprising: (a) providing a substantially continuous ring defining a ring lumen and functionally associating a membrane to the ring so that the membrane covers a portion of the ring lumen; (b) detaching at least a portion of a first a cardiac valve leaflet from a periphery of the cardiac valve in a cardiac valve including at least two cardiac valve leaflets extending from the valve periphery of the cardiac valve; (c) securing, e.g., by suturing, the substantially continuous ring to the periphery of the cardiac valve; and (d) attaching a detached edge of the cardiac valve leaflet to the membrane, thereby restoring valve function by increasing the dimensions (e.g., length and/or surface area) of the leaflet.

In some embodiments, the method further comprises, subsequent to securing (c), (e) modifying the membrane to decrease the covered portion of the ring lumen, e.g., by trimming.

In some embodiments, the membrane at least partially covers the ring lumen around the entire periphery of the ring lumen, as described above for an annuloplasty apparatus of the present invention.

In some embodiments, the cardiac valve is a bicuspid valve. In some embodiments, the cardiac bicuspid valve is a mitral valve. In some embodiments, the cardiac valve is a tricuspid valve.

In some embodiments, the leaflet is detached from the periphery substantially entirely.

In some embodiments, the attaching of the detached edge of the leaflet is proximate to a luminal edge of the membrane.

In some embodiments, prior to the attaching of the detached edge of the first leaflet, the membrane is cut so as to expose a second of the cardiac leaflets.

In some embodiments, following the attaching of the detached edge of the first leaflet, the first leaflet and the second leaflet have a length of coaptation that is greater than 8 millimeters.

In some embodiments, the attaching the detached edge of the first cardiac leaflet to the membrane includes attaching the detached edge to the membrane using a method selected from the group consisting of suturing, adhering, gluing and welding.

In some embodiments, the ring is secured by suture to the heart.

In some embodiments, the suturing is through the membrane.

In some embodiments, the membrane is shaped to cover the second cardiac leaflet.

In some embodiments, the second cardiac leaflet is retracted substantially toward the valve periphery.

In some embodiments, the cardiac valve includes at least three cardiac valve leaflets.

According to a further aspect, the present invention provides for innovative methods and implants for augmentation of the tissue surrounding a cardiac valve (e.g., the surface area of tissue between the valve annulus and the valve itself is increased). Generally, an implant including a wall, the wall delimited by two edges each in the shape of a closed curve and defining a lumen. (e.g., a tube or annulus) is provided as a cardiac valve augmenting implant. The native valve is detached from the valve annulus and secured to one edge of the implant while the other edge is secured to the valve annulus, thereby augmenting the tissue surrounding the valve. In embodiments, the implant allows distal relocation of a cardiac valve from a native position attached to a native valve annulus located between a ventricle and an atrium downwards into the ventricle.

Thus according to the teachings of the present invention there is also provided a method of augmenting the tissue surrounding a cardiac valve, comprising: a) excising leaflets of a cardiac valve (e.g., mitral valve, tricuspid valve) of a subject (human or non-human mammal) with an incision having a shape of a closed curve (e.g., circles, ovals, ellipses, oblate ovals, oblate ellipses and oblate circles), so as to define a valve seat edge of the incision and a valve periphery edge of the incision; b) providing an implant including a wall, the wall delimited by two edges each in the shape of a closed curve and defining a lumen. (e.g., a substantially tubular implant or a substantially annular implant) as a cardiac valve augmenting implant; c) securing (e.g., by suturing, adhesing, stapling) the first portion of the implant to the valve seat edge at a plurality (e.g., at least 3, generally at least 6, usually more) of locations; and d) securing (e.g., by suturing, adhesing, stapling) the second portion of the implant to the valve periphery edge at a plurality (e.g., at least 3, generally at least 6, usually more) of locations, thereby augmenting a surface area of tissue surrounding the cardiac valve with the implant, and in embodiments allowing relocation of the cardiac valve. In embodiments, spare portions of the implant are trimmed. It is important to note that the steps of the method may be performed in any rational order and not necessarily in the order listed above. For example, in embodiments, a precedes c and/or d; a succeeds c and/or d; c precedes d; d precedes c.

In embodiments, a valve (such as a mitral valve) is excised intact (that is, where the leaflets (in the case of a mitral valve, the posterior and the anterior leaflets) remain associated through the commissures from the valve annulus. In embodiments, the thus excised valve is secured to the second portion of the implant, preferably still intact.

In embodiments, the cardiac valve is a mitral valve.

In embodiments, the augmentation of the tissue surrounding the valve improves coaptation of leaflets of the cardiac valve.

As noted above, an implant used in augmenting the tissue surrounding a cardiac valve in accordance with the teachings of the present invention includes a wall, the wall delimited by two edges each in the shape of a closed curve and defining a lumen. Suitable closed curve shapes of the edges of an implant include, but are not limited to circles, ovals, ellipses, oblate ovals, oblate ellipses and oblate circles. Any suitable material or combination of materials may be used for fashioning a wall of an implant, both synthetic and biological as is detailed hereinbelow.

In embodiments, a valve augmenting implant is substantially a flat sheet of material with a hole therethrough, where the first edge is the outer edge of the flat sheet and the second edge is the edge of the hole. In such embodiments, the first region, that which is secured to the valve seat edge of the incision is a portion of the sheet closer to the first edge (edge of the sheet) than the second region which is closer to the second edge (the edge of the hole) and to which the valve periphery edge of the incision is secured. In embodiments, the flat sheet of material is in the shape of an annulus or ring. In embodiments the two edges are of the same shape. In embodiments, the two edges describe shapes that are substantially concentric.

In embodiments, augmentation of tissue surrounding the cardiac valve and subsequent relocation of a cardiac valve in accordance with the teachings of the present invention is performed with the use of a valve augmenting implant that is substantially an apparatus as described above comprising a ring including a membrane. However, instead of attaching a leaflet to the membrane, the valve is detached from a respective annulus (preferably substantially intact, that is where the leaflets are associated through substantially intact commissures) and then secured to the edge of the lumen defined by the hole in the membrane. In such embodiments, the first portion of the implant that is secured to the valve seat edge is the ring or in proximity to the ring while the second portion of the implant that is secured to the valve periphery edge is near the periphery of the hole in the membrane.

In embodiments, augmentation of tissue surrounding the cardiac valve and subsequent relocation of a cardiac valve in accordance with the teachings of the present invention is performed with the use of a substantially tubular cardiac valve augmenting implant that is substantially a tube of material having a proximal end and a distal end with a lumen passing therebetween, where the first edge is the rim of the proximal end and the second edge is the rim of the distal end. In such embodiments, the first region, that which is secured to the valve seat edge of the incision is a portion of the tube closer to the first edge (proximal rim) than the second region which is closer to the second edge (distal rim) and to which the mitral valve edge of the incision is secured. In embodiments, the tube is substantially parallel walled. In embodiments, the distal rim and the proximal rim are of substantially the same size. In embodiments, the distal end and the proximal end are coaxial. In embodiments, the distal end and the proximal end are not-coaxial. In embodiments, the proximal rim is substantially larger than the distal rim. In embodiments, the tubular wall is substantially a truncated cone. In embodiments, the distal end and the proximal end are coaxial. In embodiments, the distal end and the proximal end are not-coaxial. In embodiments, the tubular wall is substantially frustoconical. In embodiments, the ends of the truncated cone are substantially not parallel.

In embodiments, especially embodiments where the tubular cardiac valve augmenting implant is axially extensible and axially bendable, relocation of a heart valve in accordance with the teachings of the present invention allows long-term maintenance of leaflet coaptation, even in the event of continued cardiac remodeling, and reduces deformation of the valve during heart movement.

In embodiments, relocation of a cardiac valve in accordance with the teachings of the present invention is useful for restoring adequate sealing of leaky cardiac valves.

In embodiments, relocation of a cardiac valve in accordance with the teachings of the present invention is useful for restoring proper tension to improperly tensioned tendineae chordae.

Thus, according to the teachings of the present invention there is also provided a method for relocating a cardiac valve distally to a cardiac valve annulus, the method comprising: a) providing a substantially tubular cardiac valve augmenting implant comprising a substantially tubular wall defining a lumen, the implant having a proximal portion and a distal portion; b) detaching a cardiac valve from a cardiac valve annulus located between an atrium and a ventricle (e.g., mitral valve, tricuspid valve) of a subject (human or non-human mammal); c) securing (e.g., by suturing, adhesing and stapling) the cardiac valve to the distal portion of the tubular implant; and d) securing (e.g., by suturing, adhesing and stapling) the proximal portion of the tubular implant in the proximity of the cardiac valve annulus so that the valve is distal to the valve annulus, thereby providing fluid communication between the atrium and the ventricle through the lumen and through the cardiac valve.

In embodiments, securing the cardiac valve to the distal portion of the substantially tubular implant precedes the detaching of the cardiac valve from the cardiac valve annulus.

In embodiments, securing the cardiac valve to the distal portion of the substantially tubular implant is subsequent to the detaching of the cardiac valve from the cardiac valve annulus.

In embodiments, the cardiac valve is detached from the cardiac valve annulus substantially intact, for example as a complete functioning unit. For example, in embodiments, the cardiac valve is detached so that leaflets of the valve are mutually associated through substantially intact commissures of the valve.

In embodiments, the cardiac valve is secured so that at least part of the cardiac valve is located over a distal end of the substantially tubular implant

In embodiments, the cardiac valve is secured inside the lumen.

In embodiments, the cardiac valve is secured abutting against a distal end of the substantially tubular implant.

In embodiments, the cardiac valve is secured to the tubular wall.

In embodiments, the cardiac valve is secured to a ring-shaped component distinct from the tubular wall secured to the tubular wall at the distal portion of the apparatus. In embodiments, the cardiac valve is secured over a ring-shaped component distinct from the tubular wall secured to the tubular wall at the distal portion of the apparatus. Such a ring-shaped component can be considered as a prosthetic cardiac valve annulus. In embodiments, the ring-shaped component is substantially rigid. In embodiments, a first sector of the ring-shaped component is substantially rigid and a second sector of the ring-shaped component is substantially less rigid than the first sector.

In embodiments, the proximal portion of the substantially tubular implant is attached to the inner rim of the cardiac valve annulus. In embodiments, the proximal portion of the substantially tubular implant is attached above the inner rim of the cardiac valve annulus so that at least a portion of the apparatus is located over the inner rim of the cardiac annulus, for example to a portion of an inner wall of the atrium above the cardiac annulus or to a ring-shaped component (such as a prior art annuloplasty ring) located above the inner rim of the cardiac valve annulus. In embodiments, the proximal portion of the substantially tubular implant is attached below the inner rim of the cardiac valve annulus.

According to the teachings of the present invention, there is also provided a substantially tubular cardiac valve augmenting implant configured for implantation in a mammalian heart comprising: a) a substantially tubular wall defining a lumen, comprising a proximal portion with a proximal end, a distal portion with a distal end, an outer surface and a luminal surface; and b) associated with the distal end, a ring-shaped component thicker in the radial direction than the wall wherein the tubular wall is fashioned of substantially impermeable materials. Although, the method of the present invention is potentially implementable with many substantially tubular implant (for example, with a tube of tissue from an animal source), it is advantageous to implement the method of the present invention using a substantially tubular cardiac valve augmenting implant of the present invention.

Generally, the proximal portion of the tubular wall of a substantially tubular implant of the present invention is configured for attachment to a cardiac valve annulus (i.e., near the valve seat edge of the incision used to detach the cardiac valve) and functions as an extender that relocates the valve distally (i.e., lowers the valve into the ventricle).

In embodiments, a ring-shaped component associated with the distal end of the substantially tubular wall of a substantially tubular implant of the present invention functions as a prosthetic valve annulus, and in embodiments can be considered as an annuloplasty ring. In embodiments, the ring-shaped component is a prior-art annuloplasty ring associated with a substantially tubular wall.

In embodiments, at least a portion of the ring-shaped component is secured to the distal end of the substantially tubular wall by methods, including but not limited to, sewing, adhesion, gluing, suturing, riveting, stapling or welding.

The cross section of the ring (substantially perpendicular to the lumen of the ring) is of any suitable shape, including but not limited to round, oval, ovoid, square, rectangular, L-shaped and T-shaped.

In embodiments, the thickness of the ring-shaped component in the radial direction is at least about 1 millimeter, at least about 2 millimeter and even at least about 3 millimeter. In embodiments, the thickness of the ring-shaped component in the radial direction is no more than about 6 millimeter.

In embodiments, the ring-shaped component has a height of at least about 0.4 millimeter. In embodiments, the ring-shaped component has a height of no more than about 2.5 millimeter.

In embodiments, the ring-shaped component associated with the distal end of the substantially tubular wall is configured for attachment of the periphery of a cardiac valve, that is to say, the periphery of a substantially intact cardiac valve or components thereof are attachable to the ring-shaped component. In embodiments, the ring-shaped component is piercable, that is can be pierced without substantially degrading structural properties of the ring-shaped component, e.g. by sutures or staples used to secure a valve to the ring-shaped component.

In embodiments, the ring-shaped component protrudes into the lumen of the substantially tubular wall, in embodiments by at least about 1 millimeter, at least about 2 millimeter and even at least about 3 millimeter. In embodiments, the ring-shaped component protrudes into the lumen of the substantially tubular wall by no more than about 5 millimeter. In such a way, in embodiments the ring-shaped component defines a ledge to which the periphery of a cardiac valve is attachable. In embodiments, the ring-shaped component is substantially flush with the outer surface of the substantially tubular wall.

In embodiments, the ring-shaped component protrudes outwards from the outer surface of the substantially tubular wall, in embodiments by at least about 1 millimeter, at least about 2 millimeter and even at least about 3 millimeter. In embodiments, the ring-shaped component protrudes outwards from the outer surface of the substantially tubular wall, by no more than about 5 millimeter. In such a way, in embodiments the ring-shaped component defines a ledge to which the periphery of a cardiac valve is attachable. In embodiments, the ring-shaped component is substantially flush with the luminal surface of the wall.

In embodiments, the ring-shaped component is substantially flat. In embodiments, the ring-shaped component is not flat, e.g. curved.

In embodiments, the ring-shaped component describes a circle or an oblate circle. In embodiments, the ring-shaped component describes an ellipse or an oblate ellipse. In embodiments, the ring-shaped component describes an ovoid or an oblate ovoid.

In embodiments, the ring-shaped component is substantially rigid, that is substantially non-deformable both axially and radially.

In embodiments, the ring-shaped component is substantially radially non-expandable, that is, is not configured for increasing a circumference in the manner of a stent or the like. In embodiments, the ring-shaped component is substantially radially non-collapsible, that is, is not configured for decreasing a circumference in the manner of a stent or the like.

In embodiments, the ring-shaped component is substantially axially rigid.

In embodiments, the ring-shaped component is substantially flexible, that is, is deformable without changing circumference.

In embodiments, the ring-shaped component is substantially uniform, having substantially uniform properties around the circumference.

In embodiments, the ring-shaped component comprises at least two sectors, a first sector and a second sector more flexible than the first sector. In embodiments, the first sector is substantially rigid. In embodiments, the first sector is substantially flexible and the second sector even more flexible.

The ring-shaped component is fashioned of any suitable material or materials, including monolithic, woven, braided, molded, stamped and laminated materials. In embodiments, the ring shaped component comprises, essentially consists of or even consists of materials such as nitinol, stainless steel shape memory materials, metals, synthetic biostable polymer, a natural polymer, an inorganic material, titanium, pyrolytic carbon, a plastic, a titanium mesh and polydimethylsiloxane. Suitable biostable polymers include polymers such as polyolefins, polyethylenes, polytetrafluoroethylenes, polyearbonates, polyurethanes, fluorinated polyolefins, chlorinated polyolefins, polyamides, acrylate polymers, acrylamide polymers, vinyl polymers, polyacetals, polyethers, aromatic polyesters, polyetherether ketones, polysulfones, silicone rubbers, thermoset materials, polyesters and/or combinations thereof.

In embodiments, the thickness of the tubular wall is at least 0.05 millimeter at least about 0.1 millimeter and even at least about 0.2 millimeter. In embodiments, the thickness of the tubular wall is no more than about 2 millimeter, no more than about 1 millimeter and even no more than about 0.5 millimeter.

In embodiments the cross-sectional area of the lumen at the proximal end of the substantially tubular wall is less than about 28.3 cm² (equivalent to a circular lumen having a diameter of about 6 cm), less than about 19.6 cm² (equivalent to a circular lumen having a diameter of about 5 cm) and even less than about 15.9 cm² (equivalent to a circular lumen having a diameter of about 4.5 cm).

In embodiments the cross-sectional area of the lumen at the proximal end of the substantially tubular wall is greater than about 1.8 cm² (equivalent to a circular lumen having a diameter of about 1.5 cm), greater than about 3.1 cm² (equivalent to a circular lumen having a diameter of about 2 cm), greater than about 4.9 cm² (equivalent to a circular lumen having a diameter of about 2.5 cm) and even greater than about 7.1 cm² (equivalent to a circular lumen having a diameter of about 3 cm).

In embodiments, the cross-sectional area of the lumen at the proximal end of the substantially tubular wall is substantially equal to the cross-sectional area of the lumen at the distal end of the substantially tubular implant.

In embodiments, the cross-sectional area of the lumen at the proximal end of the substantially tubular implant is greater than the cross-sectional area of the lumen at the distal end of the substantially tubular implant. In embodiments, the cross-sectional area of the lumen at the distal end of the substantially tubular implant is less than about 90%, less than about 80%, less than about 70% and even less than about 60% of the cross-sectional area of the lumen at the proximal end of the substantially tubular implant.

In embodiments exceptionally suitable, for example, for implantation in a human heart, the cross-sectional area of the lumen at the proximal end of the substantially tubular implant is between about 15.9 cm² (equivalent to a circular lumen having a diameter of about 4.5 cm) and about 7.1 cm² (equivalent to a circular lumen having a diameter of about 3 cm) and the cross-sectional area of the lumen at the distal end of the substantially tubular implant is between about 5.3 cm² (equivalent to a circular lumen having a diameter of about 2.6 cm) and about 8.6 cm² (equivalent to a circular lumen having a diameter of about 3.3 cm)

In embodiments, the luminal surface is substantially smooth, allowing a smooth flow of blood through the lumen.

In embodiments, the proximal portion of the substantially tubular wall is radially expandable. In embodiments, the proximal portion of the tubular wall is radially elastic. In such a way, the proximal portion can be stretched to smoothly conform to the size of a native cardiac valve annulus

In embodiments, the substantially tubular wall is axially bendable.

In embodiments, the length (rest length, that is length in an unstressed state) of the substantially tubular wall and the ring-shaped component together is greater than about 2 millimeter and even greater than about 3 millimeter. In embodiments, the length of the substantially tubular wall and the ring-shaped component is less than about 30 millimeter, less than about 25 millimeter and even less than about 10 millimeter.

In embodiments, the substantially tubular wall is axially extensible. In embodiments, the substantially tubular wall is reversibly axially extensible and compressible. In embodiments, the substantially tubular wall is elastically axially extensible and compressible. In embodiments, the axial extensibility is from about 2 mm to about 12 mm. In embodiments, the axial extensibility is at least about 1.3 times, at least about 1.5 times and even at least about 2 times the length the of the tubular wall.

In embodiments, the substantially tubular wall is substantially radially non-expandable, that is, is not configured for increasing a circumference. In embodiments, the substantially tubular wall is substantially radially non-collapsible, that is, is not configured for decreasing a circumference.

In embodiments, the substantially tubular wall is substantially radially rigid, that is, substantially radially non-deformable.

In embodiments, the substantially tubular wall is substantially radially flexible, that is, is deformable without changing circumference.

In embodiments, the substantially tubular wall consists essentially of one material.

In embodiments, the distal portion of the substantially tubular wall consists essentially of a first material and the proximal portion of the substantially tubular wall consists essentially of a second material.

In embodiments, at least one impermeable material from which the substantially tubular wall is fashioned essentially consists of polyester (e.g., Dacron). In embodiments, at least one impermeable material from which the substantially tubular wall is fashioned essentially consists of woven polyester (e.g., Dacron).

In embodiments, at least one impermeable material comprises a tissue from an animal source. In embodiments, the tissue is selected from the group consisting of serous tissue, pericardium, pleura and peritoneum. In embodiments, the animal source is a source from the group consisting of bovine, porcine, equine and human.

In embodiments, the substantially tubular wall is radially pleated, in embodiments the radial pleating being such that the substantially tubular wall is axially bendable and substantially radially rigid, analogously to a concertina.

In embodiments, the apparatus further comprises at least one reinforcement component functionally associated with the substantially tubular wall. In embodiments, the at least one reinforcement component provides the substantially tubular wall, at least in part, with axial bendability. In embodiments, the at least one reinforcement component provides the substantially tubular wall, at least in part, with axial extensibility. In embodiments, the at least one reinforcement component provides the substantially tubular wall, at least in part, with radial rigidity.

In embodiments, at least one reinforcement component is encased within the substantially tubular wall. In embodiments, at least one reinforcement component is secured to the outside surface of the substantially tubular wall. In embodiments, at least one the reinforcement component is secured to the luminal surface of the substantially tubular wall.

In embodiments, at least one the reinforcement component comprises a helical coil coaxial with the substantially tubular wall, such as a parallel-walled or conical helical spring.

In embodiments, at least one reinforcement component comprises a reinforcement ring coaxial and associated with the substantially tubular wall. In embodiments, at least one reinforcement component comprises a series of reinforcement rings coaxial and associated with the substantially tubular wall.

The present invention also provides for the manufacture of implants such as annuloplasty apparatus and cardiac valve augmenting implants such as described herein. Thus according to the teachings of the present invention there is also provided for the use of a sheet of an implantable material for the manufacture of a cardiac valve augmenting implant, the implant including a wall comprising the material, the wall delimited by two edges each having a shape of a closed curve and defining a lumen.

In embodiments, the wall is substantially annular. In embodiments, a first edge is a periphery of the wall and a second edge is a periphery of the hole of the wall.

In embodiments, the wall is substantially tubular. In embodiments, a first edge is a periphery of a proximal end of the wall and a second edge is a periphery of a distal end of the wall.

In embodiments, the second edge is configured to be secured to an excised cardiac valve and a first edge is configured to be secured to a mitral valve seat, e.g., in proximity of a mitral valve annulus.

According to the teachings of the present invention there is also provided a method of producing a cardiac implant, comprising: a) providing a sheet of implantable material; and b) fashioning the material in the shape of a wall of the cardiac implant, the wall delimited by two edges each having a shape of a closed curve and defining a lumen.

In embodiments, the wall is substantially annular. In embodiments, a first edge is a periphery of the wall and a second edge is a periphery of the hole of the wall,

In embodiments, the wall is substantially tubular. In embodiments, a first edge is a periphery of a proximal end of the wall and a second edge is a periphery of a distal end of the wall.

In embodiments, the second edge is configured to be secured to an excised cardiac valve and a first edge is configured to be secured to a mitral valve seat.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

As used herein, the terms “comprising” and “including” or grammatical variants thereof are to be taken as specifying the stated features, integers, steps or components but do not preclude the addition of one or more additional features, integers, steps, components or groups thereof This term encompasses the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

As used herein, the indefinite articles “a” and “an” mean “at least one” or “one or more”.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 (prior art) is a schematic depiction of a healthy heart in cross section;

FIGS. 2A and 2B prior art) depict a mitral valve of a healthy heart;

FIGS. 3A and 3B (prior art) depict a mitral valve of a heart suffering from ischemic mitral regurgitation related to incomplete coaptation of the leaflets of the mitral valve;

FIG. 4 shows an aerial view of an improperly functioning mitral valve with a detached anterior leaflet, according to an embodiment of the invention;

FIGS. 5-6 show an annuloplasty apparatus being deployed in the mitral valve shown in FIG. 4, according to an embodiment of the invention;

FIGS. 7, 8A and 8B show augmentation of the anterior mitral valve leaflet using the annuloplasty apparatus shown in FIGS. 5-6, according to an embodiment of the invention; and

FIGS. 9, 10A and 10B show reconstruction of both the anterior and posterior mitral valve leaflets using the annuloplasty apparatus shown in FIGS. 5-6, according to an embodiment of the invention.

FIG. 11 depicts an aerial view of an improperly functioning mitral valve, severed from a valve annulus about the periphery of the valve so as to leave the valve leaflets associated through the commissures so that the valve is substantially intact, according to embodiments of the invention;

FIGS. 12A-12F depict various stages of an embodiment of the method of the present invention where the tissue surrounding a mitral valve such as depicted in FIG. 11 is augmented with an implant that is substantially a ring such as depicted in FIG. 5, the method leading to valve relocation downwards into the left atrium and increased leaflet coaptation;

FIG. 13 depicts a substantially tubular cardiac valve augmenting implant, according to embodiments of the invention;

FIGS. 14A and 14B depict mitral valve leaflets being attached to the valve augmenting implant of FIG. 12, according to embodiments of the invention.

FIG. 15 depicts the valve augmenting implant of FIG. 4 implanted in a heart, in cross section;

FIG. 16 depicts the valve augmenting implant of FIG. 4 implanted in a heart, in cross section subsequent to continued remodeling;

FIGS. 17A-17E, 18A-18D, 19A-19D and 20A-20C depict embodiments of the substantially tubular valve augmenting implant of the present invention;

FIG. 21 depicts an embodiment of a valve attached to a substantially tubular valve augmenting implant of the present invention;

FIGS, 22A, 22B and 22C depict embodiments of attachment of the proximal portion of a substantially valve augmenting implant of the present invention relative to a cardiac valve annulus; and

FIGS. 23A, 23B and 23C depict embodiments of ring-shaped components of substantially tubular valve augmenting implants of the present invention, in top view, cross section and perspective.

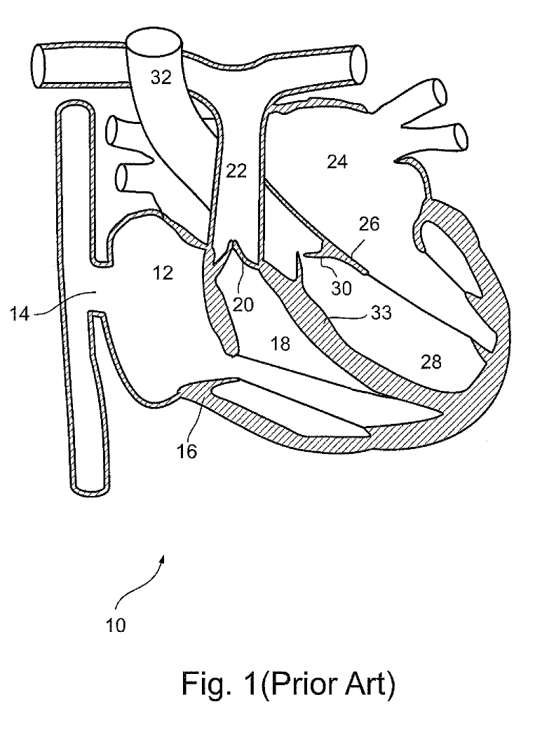

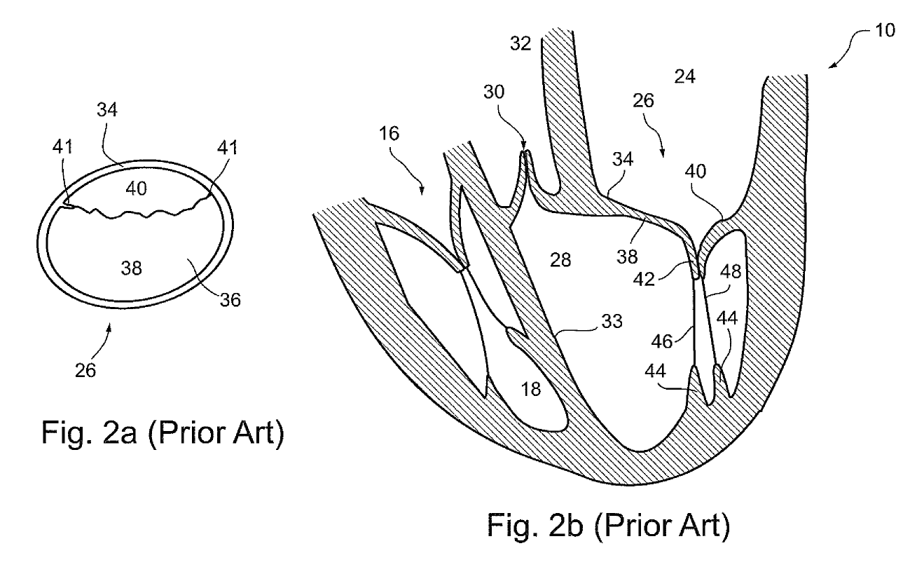

### DESCRIPTION OF EMBODIMENTS

The present invention relates to methods and devices for treatments of cardiac valves by tissue augmentation that in embodiments are useful for improving cardiac leaflet coaptation, especially of the mitral valve. Generally, according to the teachings of the present invention the subvalvular apparatus is preserved.

The principles and uses of the teachings of the present invention may be better understood with reference to the accompanying description, Figures and examples. In the Figures, like reference numerals refer to like parts throughout.

Before explaining at least one embodiment of the invention in detail, it is to be understood that the invention is not limited in its application to the details set forth herein. The invention can be implemented with other embodiments and can be practiced or carried out in various ways.

Embodiments of the present invention successfully address at least some of the shortcomings of the prior art by providing a simple method of augmenting cardiac valve leaflets. Thus, the teachings of the present invention allow a cardiac leaflet to be augmented and therefore embodiments are useful for treating a condition where cardiac valve augmentation is beneficial, such as mitral valve insufficiency, for example ischemic mitral regurgitation.

Embodiments of the present invention successfully address at least some of the shortcomings of the prior art by providing a simple method of augmenting the tissue around a cardiac valve. In embodiments, this leads to cardiac valve relocation that improves leaflet coaptation. Thus, the teachings of the present invention allow a cardiac valve to be augmented and therefore embodiments are useful for treating a condition where cardiac valve relocation is beneficial, such as mitral valve insufficiency, for example ischemic mitral regurgitation.

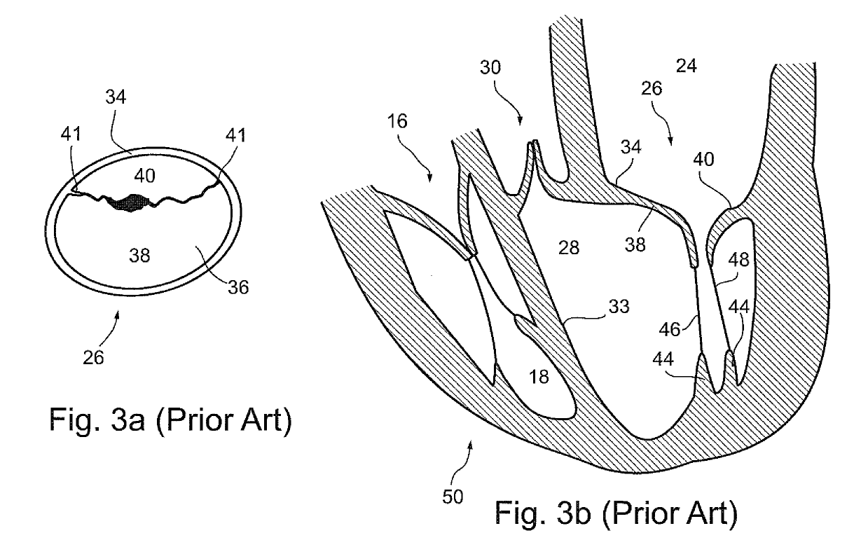

As noted above and depicted in FIGS. 3A and 3B, in a heart 50 suffering from ischemic mitral regurgitation mitral valve 26 and associated chordae 46 and 48 are patent. The insufficient coaptation of leaflets 38 and 40 that leads to the regurgitation of blood is a result of deformation of mitral valve annulus 34 and misdirected pulling forces applied through chordae 46 and 48 to leaflets 38 and 40, both resulting from necrosis and consequent deformation of the wall of left ventricle 28. In such cases, the regurgitation may be treated by improving leaflet coaptation. Embodiments of the present invention are useful in augmenting cardiac valve leaflets, especially for treating a condition where such augmentation is beneficial. Embodiments of the present invention are useful in augmenting the tissue surrounding a cardiac valve, especially for treating a condition where such augmentation is beneficial. In order to simplify understanding the teachings of the present invention embodiments of the present invention will be discussed in the context of treating a mitral valve suffering from ischemic mitral regurgitation where the teachings of the present invention are directed to increasing leaflet coaptation and thus treat the ischemic mitral regurgitation, such as mitral valve 50 depicted in FIGS. 3A and 3B.

By treating a condition is meant curing the condition, treating the condition, preventing the condition, treating symptoms of the condition, curing symptoms of the condition, ameliorating symptoms of the condition, treating effects of the condition, ameliorating effects of the condition, and preventing results of the condition.

### Leaflet Augmentation

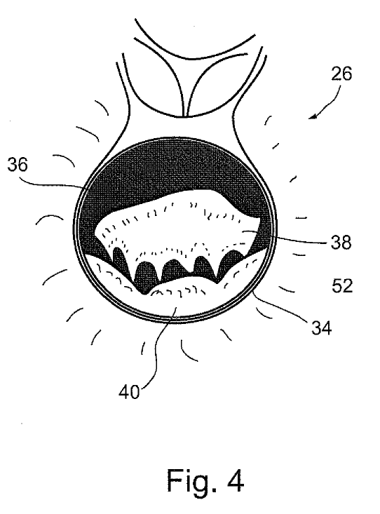

A first aspect of the present invention relates to augmentation of a cardiac leaflet, for example a posterior mitral valve leaflet. A mitral valve leaflet is detached, an annuloplasty ring with an attached membrane implanted in the substantially usual way, and the leaflet reattached to the membrane, effectively augmenting the leaflet, that in embodiments improves leaflet coaptation. An embodiment of leaflet augmentation in accordance with a method of the present invention is discussed with reference to FIGS. 4, 5, 6, 7, 5A, 8B, 9, 10A and 10B.

Referring to FIG. 4, an aerial view of a malfunctioning mitral valve 26 is shown along with mitral valve annulus 34 and adjacent left atrium floor tissue 52. Posterior leaflet 40 has been left intact while anterior leaflet 38 has been surgically incised, separated from annulus 34 and is shown floating in lumen 36.

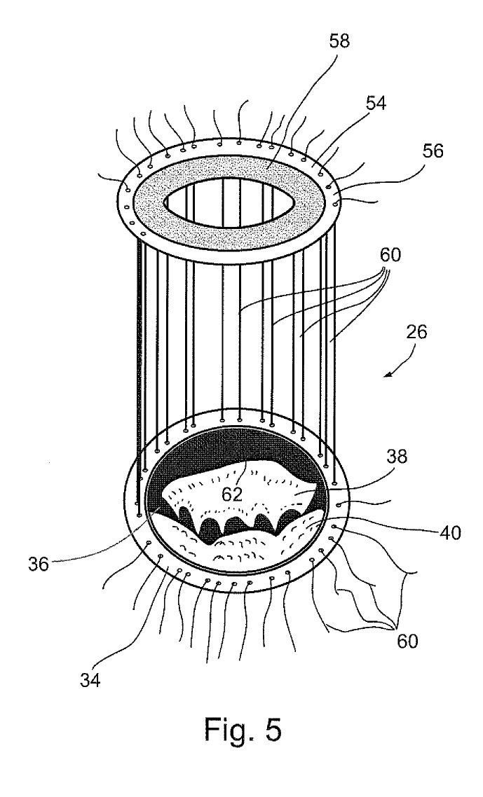

FIG. 5 shows an annuloplasty apparatus 54 of the present invention including a ring 56 and a membrane 58 substantially coplanar with ring 56. It is seen that membrane 58 partially covers the lumen of ring 56 around the entire periphery of the lumen of the ring 56.

Ring 56 may be rigid, fashioned from any one or more of various materials, for example, titanium, stainless steel, pyrolytic carbon and various plastics, as noted above. Alternatively, ring 56 may be flexible, fashioned from any one or more of various materials, including a titanium mesh, Dacron, silicon rubber, polyethylene, and polytetrafluorethylene, as noted above

Membrane 58 covers ring 56 and is configured so as to allow sutures or the like to pass through membrane 58 without substantial tearing of membrane 58, allowing annuloplasty apparatus 54 to be secured in heart tissue such as annulus 34 or in proximity thereof with sutures 60. In embodiments, annuloplasty apparatus 54 is secured to heart tissue by passing sutures 60 through membrane 58 preferably proximate to ring 56, for example through membrane 58 and looping around ring 56.

In FIG. 5, membrane 58 covers ring 56 and sutures 60 have been passed through ring 56 and through mitral valve annulus 34.

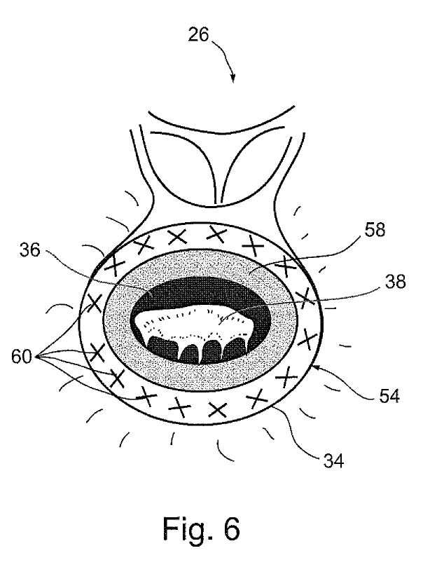

FIG. 6 shows annuloplasty apparatus 54 fully sutured to the vicinity of mitral valve annulus 34 with inverted mattress knots in sutures 60. Membrane 58 extends inwards to partially obstruct lumen 36.

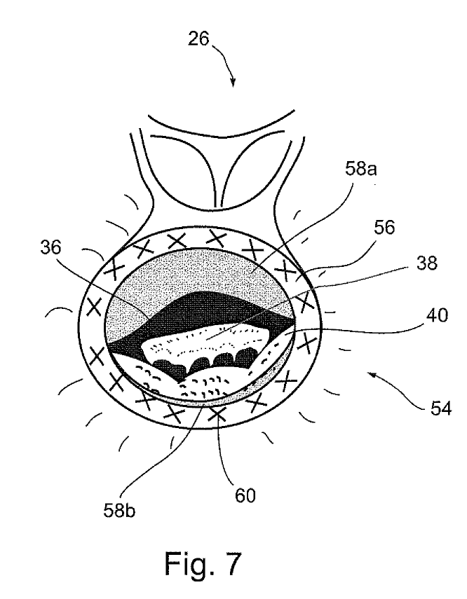

FIGS. 7 shows anterior leaflet 38 exposed along with a portion of membrane 58a that has been trimmed to be suitable for attachment of anterior leaflet 38 thereto.

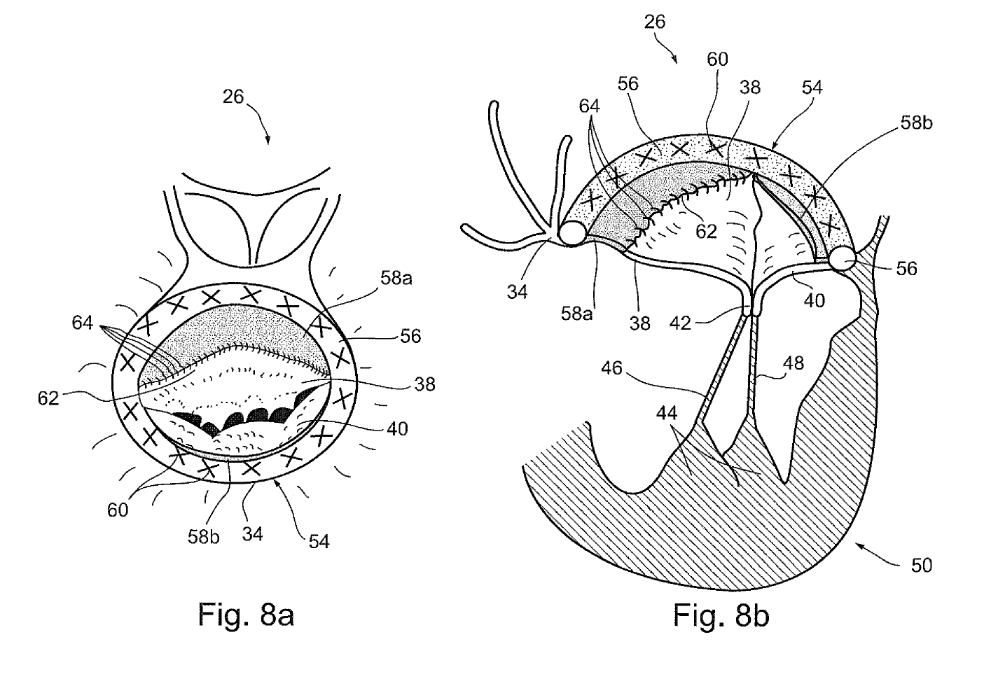

FIG. 8A shows an annular edge 62 of an anterior leaflet 38 attached to a trimmed portion 58a of membrane 58 with sutures 64.

FIG. 8B shows a cross sectional long axis view of heart 50, with annuloplasty apparatus 54 after anterior leaflet 38 has been augmented in accordance with the teachings of the present invention. Ring 56 of annuloplasty apparatus 54 is secured to the vicinity of mitral annulus 34 with sutures 60 to function substantially as a prior art annuloplasty ring. Membrane 58 of annuloplasty apparatus 54 is trimmed to two portions. Portion 58b above posterior leaflet 40 is trimmed to close with ring 56 so as not to interfere with blood flow through mitral valve 26 and proper functioning of posterior leaflet 40. Anterior leaflet 38 is secured to portion 58a of membrane 58 with sutures 64 through annular edge 62 where anterior leaflet 38 was removed from annulus 34. Portion 58a effectively augments anterior leaflet 38, increasing the surface area and the length of anterior leaflet 38. Augmentation of anterior leaflet 38 restores and increases coaptation surface 42 between leaflets 38 and 40 (compare with FIG. 3B). As depicted in FIG. 8B, coaptation surface 42 has a length of approximately 10 to 12 millimeters

It is expected that in embodiments, due to the extent of augmentation of coaptation 42 between augmented anterior leaflet 38 and posterior leaflet 40, continued remodeling of heart 50 will not result in clinically significant loss or reduction of coaptation

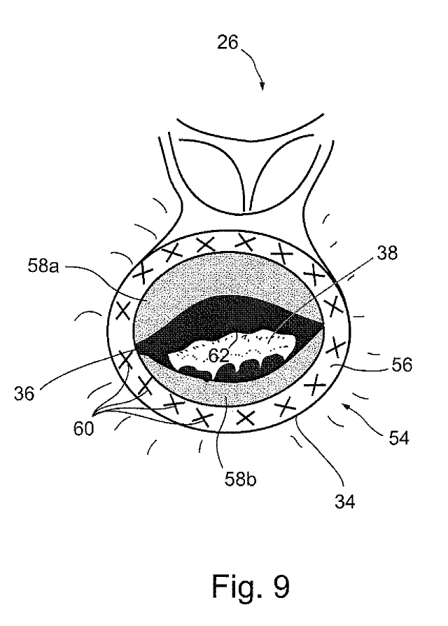

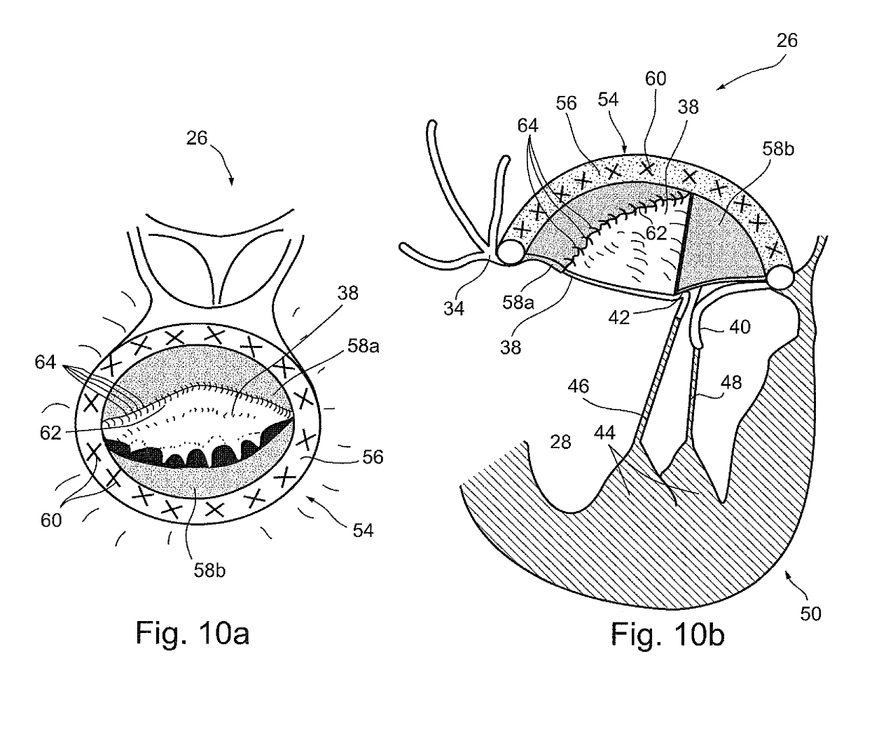

In certain pathologies, a posterior leaflet 40 is severely misaligned or, as seen in rheumatic hearts or hearts suffering from mitral annular calcification, severely misshapen. In other instances, a posterior leaflet 40 includes tissue defects, e.g., congenital defects, following debridement of endocarditis and following excision of cardiac tumors. In such cases, an annuloplasty apparatus of the present invention such as 54 is implanted in heart 50 substantially as described above but membrane 58 is trimmed substantially differently so that the portion of membrane 58 close to posterior leaflet 40 acts as a prosthetic posterior leaflet as depicted in FIGS. 9, 10 and 10B.

In FIG. 9 is seen how annuloplasty apparatus 54 is secured to mitral annulus 34 with inverted mattress sutures 60 and membrane 58 trimmed to two portions 58a proximate to anterior leaflet 38 and 58h proximate to posterior leaflet 40.

In FIG. 10A, is seen that anterior leaflet 38 is secured to portion 58a of membrane 58 with sutures 64, substantially as described above.

In FIG. 10B is seen how anterior leaflet 38 augmented with portion 58a of membrane 58 coapts with portion 58b of membrane 58 at coaptation surface 42 rather than with posterior leaflet 40.

As noted above, it is expected that in embodiments, due to the extent of augmentation of coaptation 42 between augmented anterior leaflet 38 and membrane portion 58b, continued remodeling of heart 50 will not result in clinically significant loss or reduction of coaptation

### Augmentation of Tissue Surrounding a Cardiac Valve

As noted above, an additional aspect of the present invention relates to augmentation of the tissue surrounding a cardiac valve. Generally, an implant including a wall, the wall delimited by two edges each in the shape of a closed curve and defining a lumen. (e.g., a tube or annulus) is provided as a cardiac valve augmenting implant. The cardiac valve is detached from the valve annulus and secured to one edge of the implant while the other edge of the implant is secured to the valve annulus, thereby augmenting the tissue surrounding the valve. In embodiments, the implant allows distal relocation of a cardiac valve from a native position attached to a native valve annulus located between a ventricle and an atrium downwards into the ventricle. In embodiments, such relocation alleviates the deforming effect of forces applied to the valve, for example through the valve annulus and tendineae chordae, resulting from deformation of the heart, for example due to cardiac remodeling. In embodiments, relocation of a heart valve in accordance with the teachings of the present invention increases the magnitude of leaflet coaptation by allowing for realignment of the cardiac valve leaflets (for example mitral valve leaflets), improving valve function. Some embodiments of the aspect of the invention may be useful in treating conditions, for example mitral insufficiency such as ischemic mitral regurgitation.

Augmentation of tissue surrounding a cardiac valve in accordance with the teachings of the present invention is described hereinbelow with reference to a mitral valve such as mitral valve 26 of heart 50 depicted in FIGS. 3 where the purpose of the augmentation is to restore coaptation of leaflets 38 and 40.

Using standard methods with which one skilled in the art is familiar, the subject is attached to a cardiopulmonary bypass. Heart 50 is accessed using any open surgical approach, e.g., median sternotomy, right or left thoracotomy. Alternatively, the heart is accessed using minimally invasive techniques, for example using a port access approach. The interior of heart 50 is exposed by any of several approaches, e.g., right or left sided atriotomy, transseptal incision, with or without left atrial roof opening. During repair heart 50 may be fibrillating or arrested.

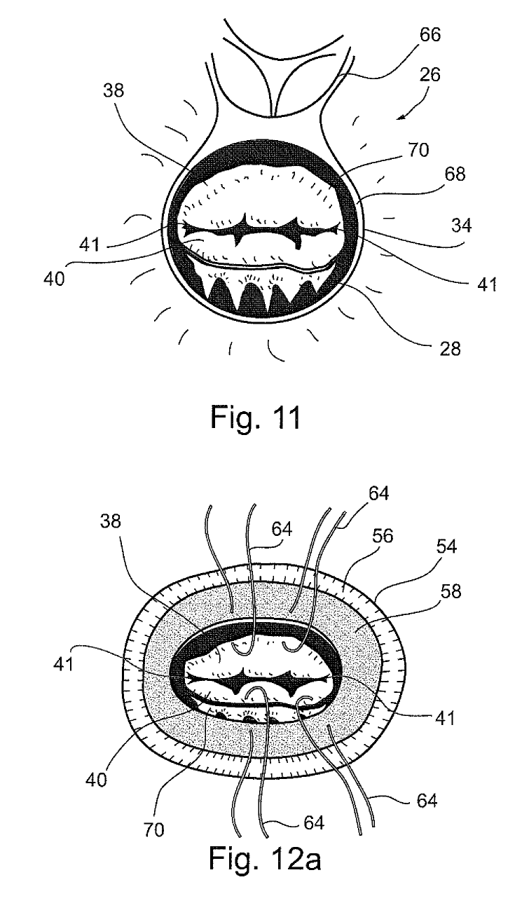

With the interior of heart 50 exposed, mitral valve 26 is detached from mitral valve annulus 34 substantially intact so as to leave leaflets 38 and 40 associated through commissures 41 so that valve 26 is floating freely within left ventricle 28 as depicted in FIG. 11. The incision that detaches mitral valve 26 from mitral valve annulus 34 defines a valve seat edge 68 and a valve periphery edge 70. For reference, annulus 34 is shown adjoining a subaortic curtain 66.

Subsequently, a cardiac valve augmenting implant is implanted, the implant including a wall, the wall delimited by two edges each in the shape of a closed curve and defining a lumen. Such implants include substantially annular implants and substantially tubular implants.

### Substantially Annular Cardiac Valve Augmenting Implant

In embodiments, augmentation of tissue surrounding a cardiac valve is performed with the use of a substantially annular cardiac valve augmenting implant. In such embodiments, a first region at or near the periphery of the wall (first edge) of the implant is secured at or near a valve seat edge 68. In such embodiments, a mitral valve 26 is secured (at or near a valve periphery edge 70 of mitral valve 26) to a second region of the implant at or near the edge of the lumen (second edge) of the implant defined by the hole in the implant.

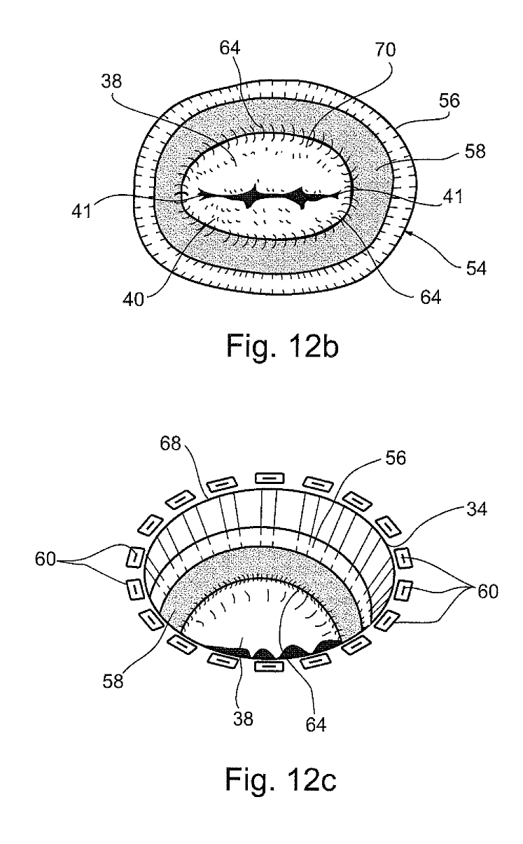

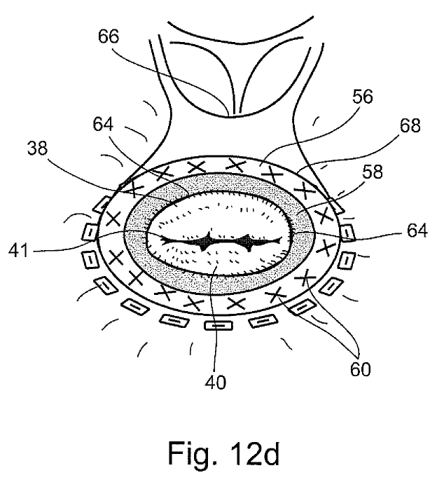

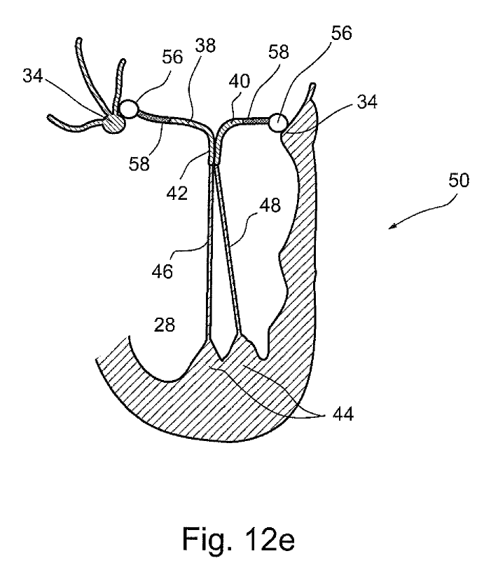

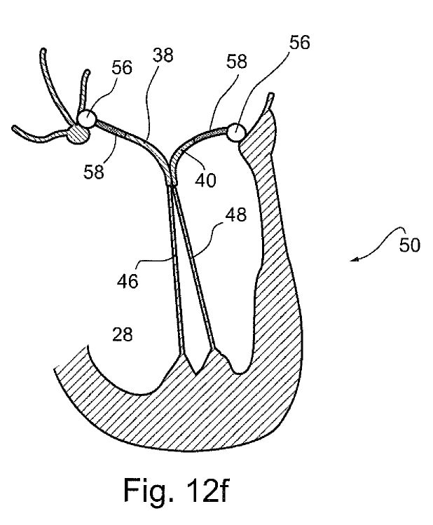

An embodiment of augmenting tissue surrounding a cardiac valve in accordance with the teachings of the present invention is discussed with reference to FIGS. 12A-12F.

As depicted in FIG. 12A, after preparing a mitral valve 26 as discussed above with reference to FIG. 11, an annuloplasty apparatus 54 is placed in heart 50 in proximity to mitral valve 26. Annuloplasty apparatus 54 is as discussed above and includes a ring 56 and a membrane 58 with a hole therethrough. Ring 56 and membrane 58 together constitute a wall of apparatus 54. The periphery of ring 56 defines the periphery of the wall of apparatus 54 which is also the first edge of apparatus 54. The rim of the hole through membrane 58 defines the second edge of apparatus 54 and thus defines the lumen of apparatus 54. Not depicted is that the hole through membrane 58 has been trimmed to a desired size to accommodate mitral valve 26. Sutures 64 are passed through mitral valve 26 near valve periphery edge 70 and through membrane 58 in a first region of membrane 58 near the periphery of the hole through membrane 58.

As depicted in FIG. 12B, sutures 64 are tightened and knotted so as to secure mitral valve 26 to membrane 58, making a strong and leak-proof seal between valve periphery edge 70 and the second edge of apparatus 54.

As depicted in FIG. 12C, sutures 60 are passed through a region of heart tissue near valve seat edge 68 and through ring 56 of apparatus 54.

As depicted in FIG. 12D, sutures 60 are tightened and knotted using inverted mattress sutures so as to secure apparatus 54 through ring 56 in proximity to valve seat edge 68, making a strong and leak-proof seal between valve seat edge 68 and the first edge of apparatus 54.

As depicted in FIG. 12E, subsequent to augmentation of tissue surrounding a cardiac valve with a substantially annular cardiac valve augmenting implant such as apparatus 54 in accordance with the teachings of the present invention, coaptation 42 of leaflets 38 and 40 is restored and or improved to a significant extent. It is expected that in embodiments, due to the extent of augmentation of coaptation 42, continued remodeling of heart 50 will not result in clinically significant loss or reduction of coaptation, as depicted in FIG. 12F.

In embodiments, a substantially annular cardiac valve augmenting implant is devoid of a ring as described above and instead is simply an annular membrane. Use and implantation of such an implant is substantially similar to the described above. In such embodiments, the valve augmenting implant is substantially a sheet of implantable material (e.g., a membrane) with a hole therethrough, where the first edge of the implant is the outer edge of the sheet and the second edge of the implant is the edge of the hole. In such embodiments, the first region, that which is secured to the valve seat edge of the incision which is a portion of the sheet closer to the first edge (edge of the sheet) than the second region which is closer to the second edge (the edge of the hole) and to which the valve periphery edge of the incision is secured. In embodiments, the flat sheet is in the shape of an annulus or ring. In embodiments the two edges are of the same shape. In embodiments, the two edges describe shapes that are substantially concentric.

### Substantially Tubular Cardiac Valve Augmenting Implant

In embodiments, augmentation of tissue surrounding the cardiac valve is performed with the use of a substantially tubular cardiac valve augmenting implant that is substantially a tube of material having a proximal end and a distal end with a lumen passing therebetween, where the first edge is the rim of the proximal end of the tube and the second edge is the rim of the distal end of the tube. In such embodiments, the first region, that which is secured to the valve seat edge of the incision is a portion of the tube closer to the first edge (proximal rim) than the second region which is closer to the second edge (distal rim) and to which the valve periphery edge of the incision is secured.

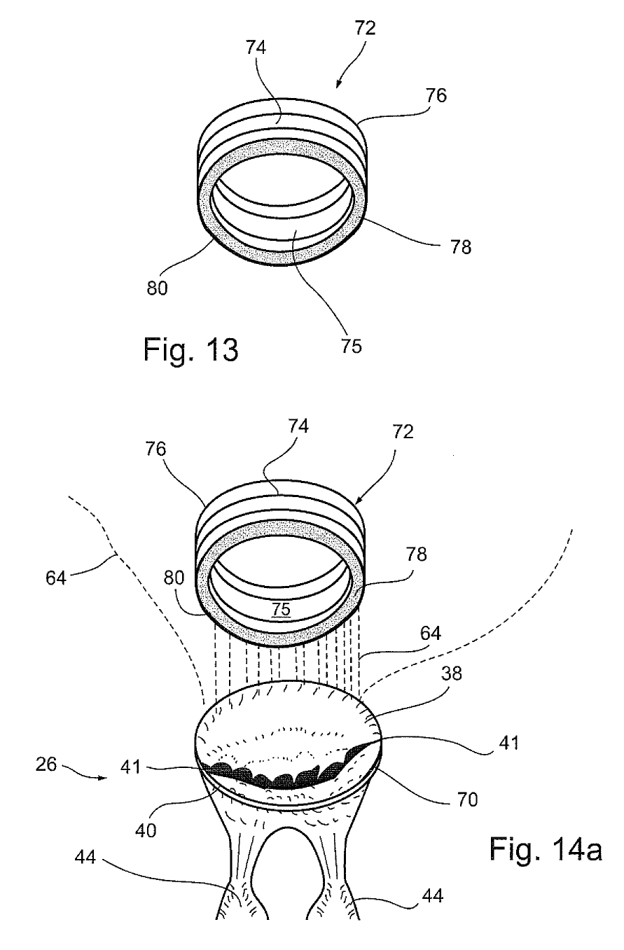

Embodiments of augmentation of tissue surrounding a cardiac valve in accordance with a method of the present invention with a substantially tubular implant is discussed with reference to FIGS. 13, 14A, 14B, 15, 16, 17A-17E, 18A-18D, 19A-19D, 20A-20C, 21, 22A-22C and 23A-23C.

FIG. 13 shows a tubular cardiac valve augmenting implant 72 of the present invention having a substantially tubular wall 74 (of impermeable pleated woven Polyester (Dacron®)) defining a lumen 75. Tubular implant 72 additionally comprises a proximal portion having a proximal end 76, and a ring-shaped component 78, a ring of titanium mesh associated with the distal end 80 of tubular wall 74 by sutures. As used herein, the terms “proximal” and “proximally” indicate an object or action located closer to mitral valve annulus 34, while “distal” and “distally” indicate an object or action located farther from annulus 34.

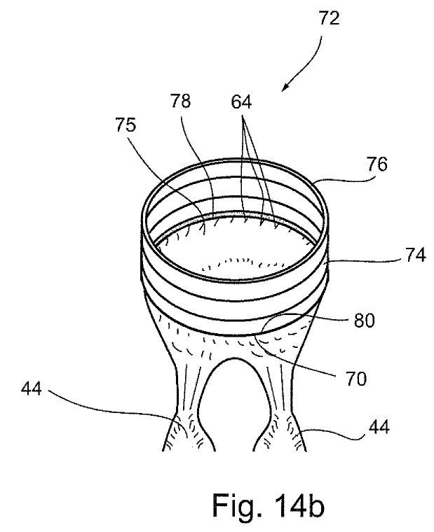

Tubular implant 72 of proper shape and size has been chosen, ring-shaped component 78 is sutured to a region near valve periphery edge 70 of mitral valve 26 as seen in FIG. 14A, using, for example, non-interrupted sutures 64 so that valve 26 abuts ring shaped component 78 at distal end 80 of tubular implant 72.

Sutures 64 are tightened so that ring-shaped component 78 and valve periphery edge 70 are in sealing contact. FIG. 14B shows valve periphery edge 70 abutting and secured to distal end 80 with sutures 64.

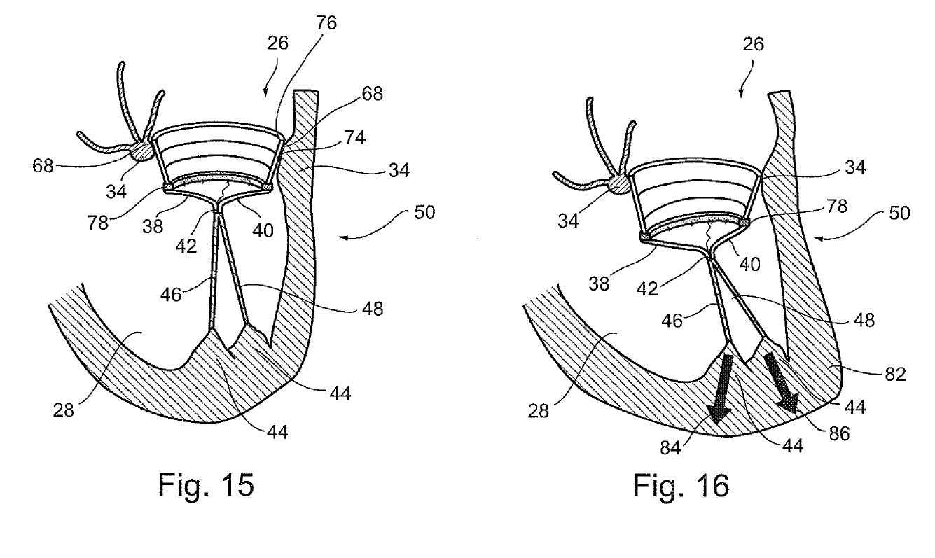

Referring to FIG. 15, prior to attaching proximal end 76 of tubular implant 72 to valve seat edge 68 in proximity of mitral valve annulus 34, the surgeon optionally measures and trims proximal end 76 of tubular wall 74 so that valve augmenting implant 72 fits properly in and does not extend above mitral valve annulus 34. The surgeon also optionally aligns valve augmenting implant 72 in mitral valve annulus 34 and observes the proper positioning of chordae tendineae 46 and 48 so that there is no impingement on leaflets 38 and 40 and verifies that coaptation surface 42 is sufficiently large.

The surgeon then secures proximal end 76 of tubular implant 72 near to valve seat edge 68 near mitral valve annulus 34 with the help of sutures. Tubular implant 72 relocates the position of leaflets 38 and 40 distally into left ventricle 28. As a result chordae 46 and 48 do not pull leaflets 38 and 40 too far downwards. In such a way, sufficient leaflet coaptation 42 is restored.

Relocation of mitral valve 26 and leaflets 38 and 40 allows the surgeon to forgo radical undermining and/or relocation of papillary muscles 44, a complex procedure that has not been effective in reducing progressive remodeling and malfunction of papillary muscles 44.

FIG. 15 shows a portion of heart 50 in a cross sectional long axis view, with leaflets 38 and 40 fully attached to tubular implant 72. Leaflets 38 and 40 are shown in the closed position during ventricular systole.

As noted above, tubular wall 74 is substantially a tube of pleated woven polyester as is known in the surgical arts for use as an arterial graft. The pleating of such a woven polyester tube provides tubular wall 74 with radial rigidity preventing collapse, deformation and obstruction of the lumen of tubular wall 74 yet provides tabular wall with axial bendability and elastic extensibility (up to about 50% of the length of tubular wall 74). This bendability and elastic extensibility of tubular wall 74 allows tubular wall 74 to adapt by bending and stretch in response to the pulling of chordae 46 and 48.

Although in embodiments, a tubular wall of a tubular valve augmenting implant of the present invention is parallel-walled so that the area of the lumen at the distal end and at the proximal end are substantially the same, in embodiments, such as tubular wall 74 of tubular implant 72, the lumen at the distal end has a smaller area than the lumen at the proximal end. Such an arrangement helps prevent entry of the tubular wall into the aorta during ventricular contraction.

FIG. 16 shows mitral valve 26 attached to ring-shaped component 78 following relocation of mitral valve 26 using tubular implant 72 as described above after a period of time where remodeling of papillary muscle ventricular wall 82 has occurred. Remodeling of wall 82 has caused papillary muscles 44 to move outwards, for example, in directions 84 and 86. Wall 74 of implant 72 stretches so that mitral valve 26 moves more distally into left ventricle 28, conforming to this motion and compensating for valvular distortion caused by remodeling thereby maintaining coaptation of leaflets 38 and 40.

As shown, cardiac wall 82 remodeling is uneven. The resultant inequality in force, however, does not cause leaflet 38 to exhibit signs of tenting, tethering, reduction of coaptation 42 and/or regurgitation. Instead, longitudinally flexible tubular wall 74 has stretched downwards and towards the left side of the heart. In embodiments, tubular wall 74 is elastically axially extensible and compressible. In embodiments, the axial extensibility is from about 2 mm to about 12 mm.

Extension of tubular wall 74 has allowed ring-shaped component 78 to tilt in a manner that equalizes the unequal pull of chordae 46 and 48 so that coaptation surface 42 is maintained.

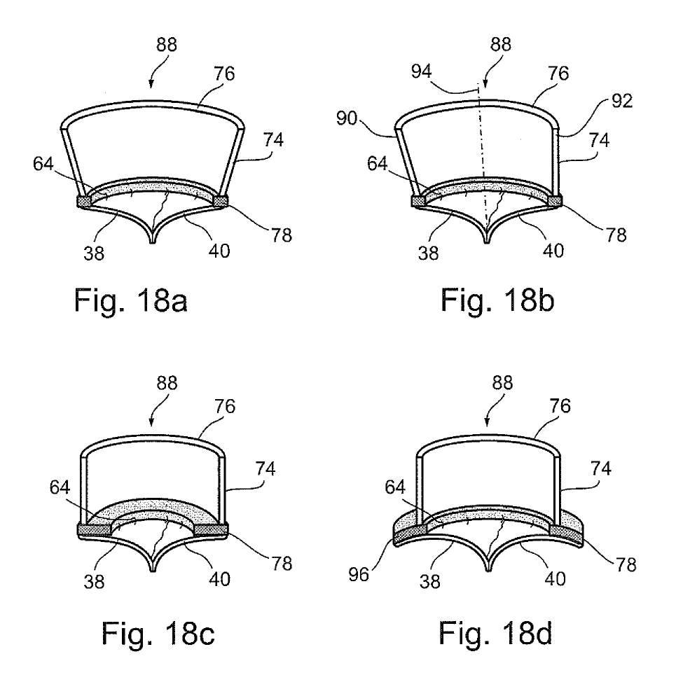

In embodiments, (seen FIG. 18C) wall 74 is substantially non-stretchable and ring-shaped component 78 extends into lumen 88 by anywhere from 5 to 15 millimeters.

In embodiments (as discussed with reference to FIG. 15), the proximal end of the tubular wall is trimmable, that is, can be shortened by a desired extent without adversely affecting the functioning of the tubular implant. In embodiments, prior to attachment of the proximal end of the tubular wall to the vicinity of the cardiac annulus, the proximal portion of the tubular wall is trimmed so that the height of leaflet coaptation surface 42 is set to between 10 and 15 millimeters, ensuring that leaflets 38 and 40 will properly coapt and that regurgitation through leaflets 38 and 40 will not recur, even in the face of post-operative remodeling of ventricular wall 82 (FIG. 16) and the pull of papillary muscles 44.

In embodiments, the tubular wall of an implant is secured to the vicinity of the cardiac valve annulus at a location along the wall to provide a desired degree of leaflet coaptation, and subsequently excess tubular wall that extends into the atrium is trimmed.

In exemplary embodiments, tubular implant 72 is provided in various sizes and shapes that depend, inter alia, on the diameter and/or shape of mitral valve annulus 34 (FIG. 16) and/or the valve periphery edge 70 and whether there is a necessity to alter the shape of mitral valve 26 and/or leaflets 38 and 40.

As a non-limiting example, the surgeon may choose a tubular implant having a diameter of proximal end 76 of 28 millimeters. In a tubular implant 72 having a tubular wall 74 that is substantially parallel to a longitudinal axis passing through lumen 88, ring 78 will have an effective orifice area of 480 millimeters².

In some instances, the surgeon opts to reduce the native diameter of valve periphery edge 70 in order to increase coaptation of leaflets 38 and 40. In some embodiments, tubular wall 74 is sloped along its entire outer surface, thereby reducing the cross section of lumen 88 of the tubular implant at ring-shaped component 78.

As a non-limiting example, the surgeon may choose a tubular implant having a tubular wall diameter of 28 millimeters at proximal end 76 while lumen 88 of the tubular implant, as measured at ring-shaped component 78, has a smaller diameter, thereby reducing effective orifice area to 466 millimeters², as seen in FIG. 18A. Upon attachment of mitral valve 26, the diameter of valve periphery edge 70 will be reduced, thereby increasing coaptation of leaflets 38 and 40.

In other embodiments, as seen in FIG. 18B, a side of tubular wall 90 is sloped with respect to a proximal portion 76 while opposite wall side 92 is substantially parallel to a luminal axis 94, thereby reducing and offsetting ring-shaped component 78 and leaflets 38 and 40.

In other embodiments (e.g., 18C), a ring-shaped component 78 projects radially inward into lumen 88, thereby providing a lip or ledge for attachment components such as sutures 64, so the attachment of a mitral valve 26 to ring-shaped component 78 is within lumen 88.

Alternatively, ring-shaped component 78 comprises a flexible distal lip 96, as seen in FIG. 18D, that deflects into lumen 88 during securing, and retracts out of lumen 88 following attachment to the tubular implant.

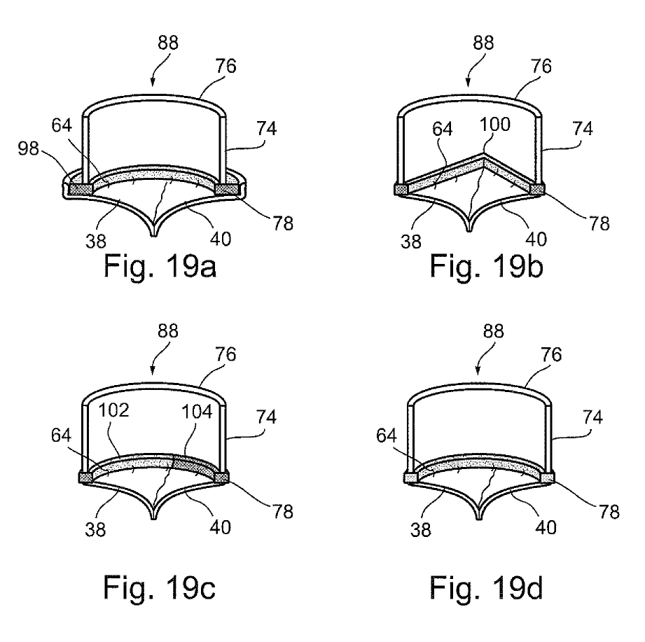

In other embodiments, a ring-shaped component 78 includes a projection 98 that projects radially outward from tubular wall 74, as seen in FIG. 19A, to enhance the ease of placing securing components such as sutures.

In still other embodiments, a ring-shaped component 78 includes a bend 100, as seen in FIG. 19B, for example: to compensate for tenting of either leaflet 38 or leaflet 40.

Many different configurations of a ring-shaped component 78 may be conceived by one skilled in the art upon perusal of the description herein.

There are many configurations of materials, material properties and attachment methods between a tubular wall 74 and a ring-shaped component 78 which may be conceived by one skilled in the art upon perusal of the description herein.

Described above have been ring-shaped components that are substantially uniform, that is the extent of rigidity or flexibility, was well as other properties is substantially at all locations about the ring-shaped component.

In embodiments, the ring-shaped component comprises at least two sectors, a first sector and a second sector more flexible than the first sector. In embodiments, the first sector is substantially rigid. In embodiments, the first sector is substantially flexible and the second sector is even more flexible. Such a configuration is known, for example, in the field of annuloplasty, where it is known that a sector of a ring close to an anterior leaflet 38 is preferably more flexible than a sector of a ring close to a posterior leaflet 40. For example, in FIG. 19C, ring 78 comprises two sectors: a rigid sector 102, for example comprising a solid metal; and a more flexible sector 104, for example comprising a metal mesh. Many combinations of material properties and configurations that are optionally used in a ring such as 78 may be conceived by one skilled in the art upon perusal of the description herein. In some embodiments, such as in FIG. 19D, ring 78 is of a uniformly flexible material.

In embodiments, following full excision of mitral valve 26 from valve annulus 34, a properly configured stapler is used to attach the valve to a ring-shaped component 78. For example, a Proximate Prolapse and Hemorrhoids (PPH) Stapler by Johnson and Johnson (not shown) may be used to staple a valve periphery edge 70 to a ring-shaped component 78.

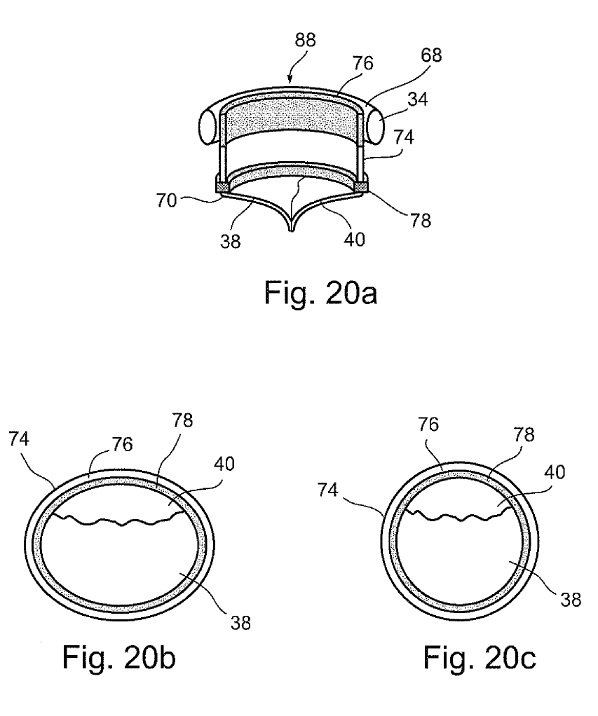

When ring 78 is substantially oval (FIG. 20B), the stapler gently bends oval ring-shaped component 78 into a circle (FIG. 20C) during stapling. Upon removal of the stapler, oval ring 78 returns to oval shape (FIG. 20B). To allow oval-to-circular-to-oval transposition, such a ring-shaped component 78 optionally comprises a semi-rigid material, for example a metal mesh.

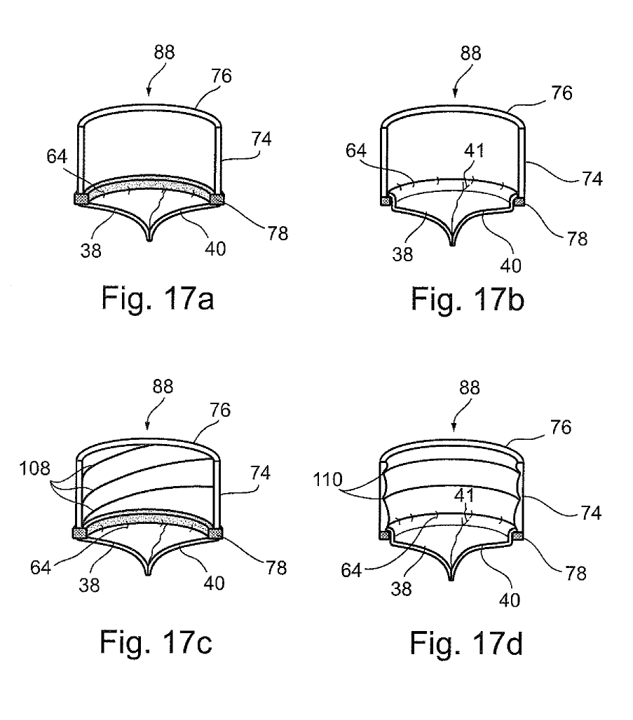

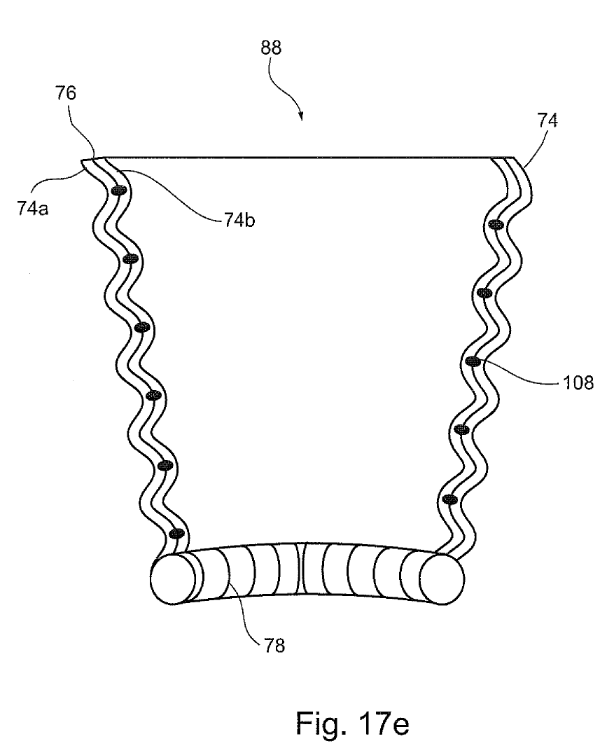

In embodiments, a cardiac valve is secured inside the lumen of a tubular wall as depicted in FIG. 17B and 17D. In embodiments, the cardiac valve is secured over a distal end of the tubular implant as depicted in FIG. 19A. In embodiments, the cardiac valve is secured abutting against a distal end of the tubular implant as depicted in FIGS. 17A, 17C, 18A, 18B, 18C, 18D, 19B, 19C, 19D, 20A and 20C

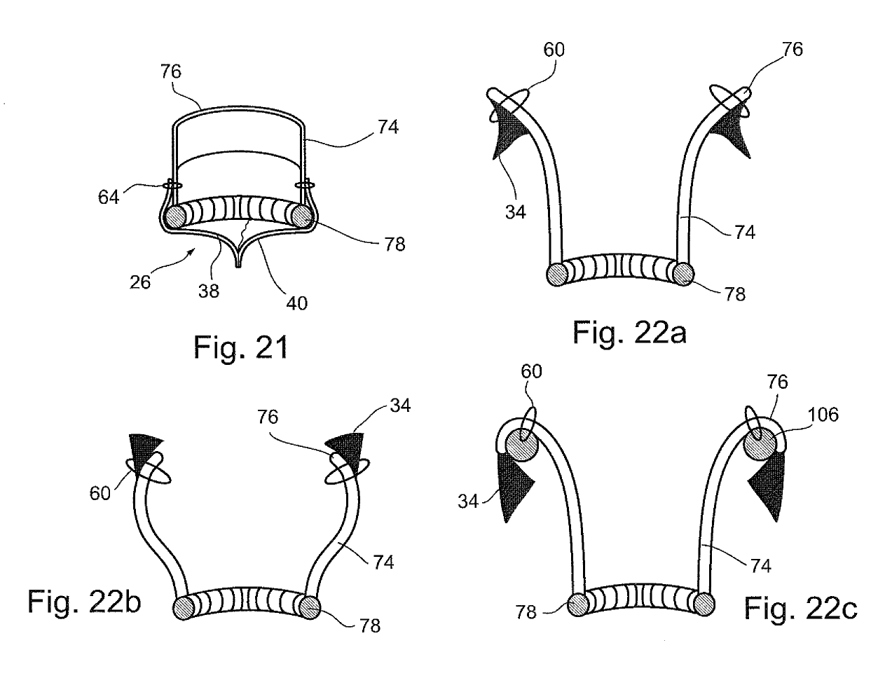

In embodiments, a cardiac valve 26 is secured to the tubular wall 74, as depicted in FIG. 21, for example with sutures 64.

In embodiments, the proximal portion 76 of a tubular wall 74 is attached to the inner rim of the cardiac valve annulus 34, as depicted in FIG. 15 or FIG. 20A. As depicted in FIGS. 22A and 22C, in embodiments the proximal portion of the tubular wall 74 is attached above the inner rim of the cardiac valve annulus 34 so that at least a portion of the implant is located over the inner rim of the cardiac annulus 34, for example to a portion of an inner wall of the atrium 24 above the cardiac annulus 34 (FIG. 22A) or to a ring-shaped component 106 (such as a prior art annuloplasty ring) located above the inner rim of the cardiac valve annulus 34 (FIG. 22C). In embodiments, the proximal portion 76 of the tubular wall 74 of the tubular implant is attached below the inner rim of the cardiac valve annulus 34, FIG. 22B.

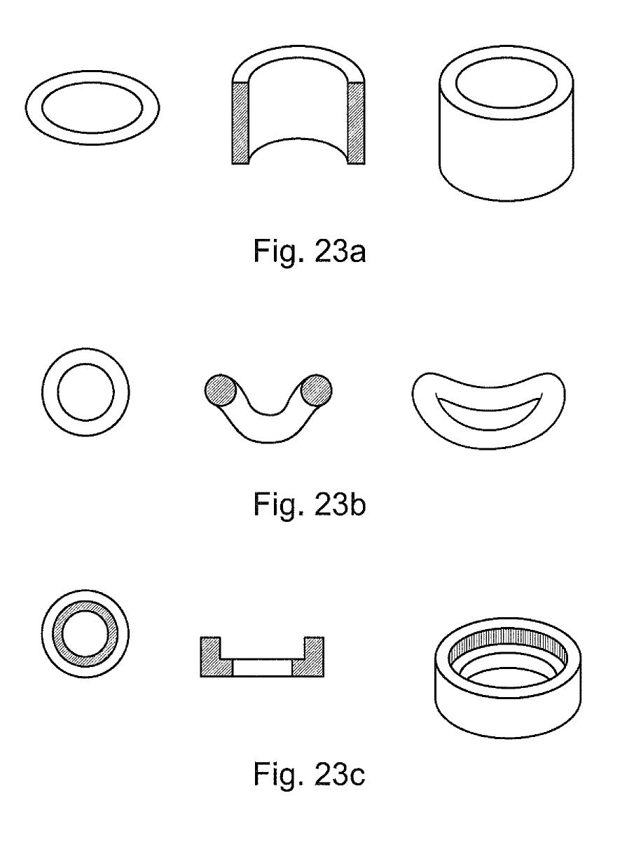

As discussed hereinabove, many different shapes of ring-shaped components 78 are suitable for implementing the teachings of the present invention. In addition to the above, in FIG. 23A is depicted a ring-shaped component having a rectangular cross-section that describes an ellipse. In FIG. 23B is depicted a ring-shaped component having a circular cross-section that describes a circle that is bent and is not flat. In FIG. 23C is depicted a flat ring-shaped component having an L-shaped cross-section that describes a circle.

In embodiments, the cross-sectional area of the lumen at the proximal end is substantially equal to the cross-sectional area of the lumen at the distal end, for example, as depicted in FIGS. 17A-17D. In embodiments, the cross-sectional area of the lumen at the proximal end is greater than the cross-sectional area of the lumen at the distal end, as depicted in FIGS. 18A and 18B.

In embodiments, such as depicted in FIG. 17D, secured to the luminal surface (in non-depicted embodiments, secured to the outer surface) of the tubular wall (fashioned of woven polyester) is a series of rings or hoops 110 (e.g., of rigid titanium or nitinol wire) as reinforcement components, arranged coaxially with the axis tubular wall. The series of loops provide the tubular wall with radial rigidity and also allow axial bendability without kinking or folding that would otherwise obstruct the lumen of the tubular wall. In embodiments, the rings flexibly elastic so as to provide a radial flexibility, that is allow elastic radial deformation without changing circumference or allowing collapse of the lumen. In FIG. 17C, reinforcement component 108 is a conical section helical spring.

Embodiments, such as depicted in FIG. 17E, are provided with a conical section helical spring 108 (e.g., of titanium or nitinol wire) as a reinforcement component encased within tubular wall 74. Tubular wall 74 comprises two layers 74a and 74b of serous tissue (peritoneum) with the respective basement layers facing each other and sandwiching helical spring 108 therebetween, mutually secured with biological glue or other suitable adhesive. In such a way, the smooth serous layer of the serous tissue face outward in contact with blood while the tough basement layers hold helical spring 108. Helical spring 108 is sandwiched and glued between the serous layers when slightly lengthened and released only when dry so as to bias the entire construct to a shortened configuration, substantially pleating the serous tissue. In such a way, helical spring 108 provides, in part, not only radial flexibility as described above, but also both axial extensibility and axial bendability to the tubular wall. Secured to the distal end of tubular wall 74 (by sutures) and engaging of the end of helical spring 108 is a slightly flexible and piercable ring-shaped component 78 of titanium mesh.

In most of the embodiments discussed above, the teachings of the present invention have been discussed where a mitral valve is relocated by implantation of a cylindrical tubular implant where the distal end and the proximal end of the tubular wall are substantially of similar size and shape. In embodiments, implants having tubular walls with other shapes are implanted including tubular implants that are frustoconical (distal and proximal ends are not parallel).

In embodiments where the teachings of the present invention are applied to augmenting the tissue surrounding a mitral valve it is important that subsequent to deployment of the implant, the mitral valve has a mitral lumen large enough to allow passage of sufficient blood. It is important to note that a person weighing between 60 and 100 kg has a usual cardiac output of about 4 to 6 l blood/minute and about 15 l blood/minute during maximum effort. It is known that a mitral valve lumen having a diameter of at least about 28 mm diameter is needed to transfer 15 l blood minute without undue stress. Thus, generally it is desirable that the implant be configured so that the diameter of the mitral valve lumen subsequent to implantation be at least about 28 mm in diameter. For example, in embodiments the edge of the implant to which the valve edge is secured is at least about 28 mm in diameter.

In the embodiments described above, the cardiac (e.g., mitral) valve is first detached from the respective annulus, and then secured to an edge of an implant of the present invention. In embodiments, a cardiac valve is first secured to an edge of an implant and then detached from the respective annulus.

In the embodiments described above, the cardiac (e.g., mitral) valve is detached from the respective annulus substantially intact as a complete functioning unit where the leaflets of the valve are mutually associated through commissures of the valve as depicted in FIG. 11. Such embodiments are exceptionally simple to implement. In embodiments, the cardiac valve is detached not intact, for example, each leaflet separately. In such embodiments, for example, each leaflet is secured to the edge of the implant separately. Such embodiments allow repair or replacement of a damaged leaflet.

When implementing the teachings of the present inventions, the membranes of an annuloplasty apparatus or the walls of a cardiac valve augmenting implants, whether as sheets with holes, annuli, tubes or other, may comprise any suitable material or combination of materials, whether synthetic or biological. Preferably at least one material from which an implant is fashioned is impermeable to prevent the flow of blood through the implant once implanted. Typically, the thickness of the tubular wall is at least 0.05 millimeter at least about 0.1 millimeter and even at least about 0.2 millimeter. Typically, the thickness of the tubular wall is no more than about 2 millimeter, no more than about 1 millimeter and even no more than about 0.5 millimeter.

Typical synthetic materials suitable for fashioning a membrane of an annuloplasty apparatus or a wall of a cardiac valve augmenting implant of the present invention include but are not limited to fluorinated hydrocarbons such as polytetrafluoroethylene, urethane, elastomer, polyamide, polyethylene, polyester (e.g., Dacron®), silicon rubber and titanium mesh.

Sources of typical biological materials suitable for fashioning a membrane of an annuloplasty apparatus of a wall of a cardiac valve augmenting implant of the present invention include but are not limited to materials from a human source, an equine source, a porcine source or a bovine source. In embodiments, biological materials used for fashioning an implant of the present invention include but are not limited to autologous tissue, homologous tissue and heterologous tissue. Specific examples include venous tissue, arterial tissue, serous tissue, dura mater, pleura, peritoneum, pericardium and aortic leaflet. In embodiments, the tissue is toughened, for example by crosslinking in the usual way.

The present invention also provides for the manufacture of implants such as annuloplasty apparatus and cardiac valve augmenting implants such as described herein. Thus according to the teachings of the present invention there is also provided for the use of a sheet of an implantable material (as described hereinabove) for the manufacture of a cardiac valve augmenting implant, the implant including a wall comprising the material, the wall delimited by two edges each having a shape of a closed curve and defining a lumen.

In embodiments, the wall is substantially annular. In embodiments, a first edge is a periphery of the wall and a second edge is a periphery of the hole of the wall.

In embodiments, the wall is substantially tubular. In embodiments, a first edge is a periphery of a proximal end of the wall and a second edge is a periphery of a distal end of the wall.

In embodiments, the second edge is configured to be secured to an excised cardiac valve and a first edge is configured to be secured to a mitral valve seat, e.g., in proximity of a mitral valve annulus.

According to the teachings of the present invention there is also provided a method of producing a cardiac implant, comprising: a) providing a sheet of implantable material (as described hereinabove); and b) fashioning the material in the shape of a wall of the cardiac implant, the wall delimited by two edges each having a shape of a closed curve and defining a lumen.

In embodiments, the wall is substantially annular. In embodiments, a first edge is a periphery of the wall and a second edge is a periphery of the hole of the wall.

In embodiments, the wall is substantially tubular. In embodiments, a first edge is a periphery of a proximal end of the wall and a second edge is a periphery of a distal end of the wall.

In embodiments, the second edge is configured to be secured to an excised cardiac valve and a first edge is configured to be secured to a mitral valve seat.

While the description of methods and apparatus of the invention have been directed to restoring proper function to mitral valves, it will be clear to those familiar with the art, that the methods and apparatus are also applicable to restoring proper function to a tricuspid valve (not shown), in some cases with minor modification which one skilled in the art is able to formulate upon perusal of the specification.

Further, while the description of methods and apparatus were directed to improperly functioning mitral valves with dysfunction of papillary muscle wall, it will be clear to those familiar with the art, that the methods and apparatus are also applicable to any disorder causing improper closure of mitral valve including, inter alia: mitral valve prolapse; rheumatic heart disease; mitral annular calcification; cardiac tumors; congenital defects; endocarditis; atherosclerosis; hypertension; left ventricular enlargement; connective tissue disorders such as Marfan's syndrome; and untreated syphilis.

The various embodiments of the present invention, especially the methods of augmenting tissue, have been described herein primarily with reference to treatment of living human subjects. It is understood, however, that embodiments of the present invention are performed for the veterinary treatment of a non-human mammal, especially horses, cats, dogs, cows and pigs.

The various embodiments of the present invention, especially the methods of augmenting tissue, have been described herein primarily with reference to treatment of living subjects. It is understood that application of the present invention for training and educational purposes (as opposed to treating a condition) falls within the scope of the claims, whether on a living non-human subject or on a dead subject, whether on a human cadaver or on a non-human body, whether on an isolated cardiac valve, or on a valve in a heart isolated (at least partially) from a body, or on a body.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-23. (canceled)

24. A cardiac valve augmenting implant comprising:
a) a substantially tubular and flexible wall, smooth on an inside and an outside of said wall for allowing a smooth flow of blood, defining a lumen, comprising a proximal portion with a proximal end with a cross-sectional area greater or equal to a cardiac valve, a distal portion with a distal end with a cross-sectional area equal to a cardiac valve, an outer surface and a luminal surface; and
b) associated with said distal end, a flexible ring-shaped component thicker in the radial direction than said wall,
configured for implantation in a human adult heart.

25. The implant of claim 24, wherein said proximal portion of said tubular wall is configured for attachment to a cardiac valve annulus.

26. The implant of claim 24, wherein said ring-shaped component is configured for attachment of the periphery of a cardiac valve.

27. The implant according to claim 24, wherein said proximal portion of said tubular wall is radially expandable.

28. The implant according to claim 24, wherein said tubular wall is axially bendable.

29. The implant according to claim 24, wherein said tubular wall is axially extensible.

30. The implant according to claim 24, wherein said tubular wall is substantially radially non-expandable.

31. The implant according to claim 24, wherein said tubular wall is substantially radially non-collapsible.

32. The implant of claim 24, further comprising at least one reinforcement component functionally associated with said tubular wall.

33. A method for relocating a cardiac valve distally to a cardiac valve annulus, the method comprising:
a) providing a substantially tubular and flexible implant comprising a substantially tubular wall, smooth on an inside and an outside of said wall for allowing a smooth flow of blood, defining a lumen, said apparatus having a proximal portion with a cross-sectional area greater or equal to a cardiac valve and a distal portion with a cross-sectional area equal to a cardiac valve;
b) detaching a cardiac valve from a cardiac valve annulus located between an atrium and a ventricle of a subject;
c) securing said cardiac valve to said distal portion of said tubular implant; and
d) securing said proximal portion of said tubular implant in the proximity of said cardiac valve annulus so that said valve is distal to said valve annulus,
thereby providing fluid communication between said atrium and said ventricle through said lumen and through said cardiac valve.

34. The method according to claim 33, wherein said cardiac valve is detached substantially intact.

35. The use of a sheet of implantable material for the manufacture of a cardiac valve augmenting implant, said implant including a wall comprising said material, said wall delimited by two edges each having a shape of a closed curve and defining a lumen.

36. The use of claim 35, wherein said wall is substantially annular.

37. The use of claim 36, wherein a first said edge is a periphery of said wall and a second said edge is a periphery of a hole of said wall.

38. The use of claim 35, wherein said wall is substantially tubular.

39. The use of claim 38, wherein a first said edge is a periphery of a proximal end of said wall and a second said edge is a periphery of a distal end of said wall.

40. The use of claim 35, wherein a second said edge is configured to be secured to an excised cardiac valve and a first said edge is configured to be secured to a mitral valve seat.

41. A method of producing a cardiac implant, comprising:
a) providing sheet of implantable material smooth on both sides of said sheet for allowing a smooth flow of blood; and
b) fashioning said material in the shape of a wall of the cardiac implant said wall delimited by two edges each having a shape of a closed curve and defining a lumen.

42. The method of claim 41, wherein said wall is substantially annular.

43. The method of claim 42, wherein a first said edge is a periphery of said wall and a second said edge is a periphery of a hole of said wall.

44. The method of claim 41, wherein said wall is substantially tubular.

45. The method of claim 44, wherein a first said edge is a periphery of a proximal end of said wall with a cross-sectional area greater or equal to a cardiac valve and a second said edge is a periphery of a distal end of said wall with a cross-sectional area equal to a cardiac valve.

46. The method of claim 41, wherein a second said edge is configured to be secured to an excised cardiac valve and a first said edge is configured to be secured to a mitral valve seat.

47. The implant according to claim 24 in which the tubular wall is substantially a truncated cone.

48. The implant of claim 24, in which said ring-shaped component is attached to the periphery of a cardiac valve

49. The method of claim 41, wherein said wall is substantially a truncated cone.

50. The use of claim 35, wherein said wall is substantially a truncated cone.
